(19) United States
(12) Patent Application Publication
(10) Pub. No.: US 2025/0235415 A1
(43) Pub. Date: Jul. 24, 2025

(54) MODULATION OF HUMAN BREAST MILK COMPOSITION

(71) Applicant: LUNDQUIST INSTITUTE FOR BIOMEDICAL INNOVATION AT HARBOR-UCLA MEDICAL CENTER, Torrance, CA (US)

(72) Inventors: Michael G. ROSS, Torrance, CA (US); Mina DESAI, Torrance, CA (US)

(21) Appl. No.: 18/840,042

(22) PCT Filed: Feb. 22, 2023
(86) PCT No.: PCT/US2023/013620

§ 371 Date: Aug. 20, 2024

Related U.S. Application Data
(60) Provisional application No. 63/313,272, filed on Feb. 23, 2022.

(51) Int. Cl. A61K 31/155; A61K 45/06

(52) U.S. Cl. CPC A61K 31/155; A61K 45/06

(57) ABSTRACT

Compositions and methods for improving a child, such as an infant's health, are provided. The methods entail improving the quality of breast milk in a female human individual that provides breast milk, or expects to provide breast milk, to the child, by administering to the individual an agent that increases the individual's sensitivity to insulin, and/or an agent that reduces the substrate uptake, synthesis or secretion of long chain fatty acids, reduces the substrate uptake, synthesis or secretion of short chain fatty acids, increases the amino acid uptake, protein synthesis or protein secretion of proteins, or reduces the uptake or synthesis of lactose.

### CROSS REFERENCE TO RELATED APPLICATIONS

This application claims the benefit under 35 U.S.C. § 119 (e) of the U.S. Provisional Application Ser. No. 63/313,272, filed Feb. 23, 2022, the content of which is hereby incorporated by reference in its entirety.

### GOVERNMENT LICENSE RIGHTS

This invention was made with government support under R01HD099813 and R21HD104028 awarded by the National Institutes of Health, Eunice Kennedy Shriver National Institute of Child. The government has certain rights in the invention.

### BACKGROUND

Nearly 80% of women initiate breastfeeding their babies at birth, and over a quarter of infants are still in part breastfed at 12 months. For the infant, breastfeeding reduces the risk of developing associated diseases (respiratory, gastrointestinal, and ear infections) as compared to formula feeding. The American Academy of Pediatrics and the WHO recognize exclusive human milk feeding as the “normative standards for infant feeding” for the first six months after birth.

Human milk is a highly variable and complex biological fluid that contains more than 200 identified components within compartments of true solutions, colloids (casein micelles), membranes, membrane-bound globules, and live cells. Constituent categories include aqueous vs. lipid fractions and nutritive vs. nonnutritive constituents. On average, the human milk contains 3-5% of fat, 0.8-0.9% of protein, 6.9-7.2% of carbohydrates, and 0.2% of minerals. Human milk median caloric content is 66 kcal/100 mL with an interquartile range of 62.0 to 72.5 kcal/100 mL, reflecting a significant individual variance. Triglycerides (TGs) make up 98% of milk lipid content and contribute 40-50% of human milk energy content. The variability in milk fatty acid (FA) composition contributes to the variance in milk caloric content.

Human milk TGs result from three sources: endogenous fat stores (adipose tissue and liver), dietary lipids, and de novo synthesis by mammary epithelial cells (MECs). Milk long-chain FAs are derived from either diet or endogenous fat stores, as human MECs have limited ability to synthesize C18 FAs. These long-chain FA which primarily are carried by very low density lipoproteins arc taken up by MECs following hydrolysis by lipoprotein lipase (LPL). MEC lipid secretion depends on the abundance of gene transcripts encoding key lipogenic regulatory factors including sterol response element binding protein 1 (SREBP1) and peroxisome proliferator-activated receptors (PPARγ). In contrast to long-chain FAs, MECs produce short- and medium-chain FAs (C6-C14) de novo with acetate (and a contribution of β-hydroxybutyrate) as the primary carbon source being absorbed through the MEC basolateral membrane. Their synthesis is primarily catalyzed by fatty acid synthase (FAS) and acetyl CoA carboxylase, while acylthioesterase serves to terminate the elongation of FAs. The lactogenic hormones (prolactin, insulin, cortisol) directly activate the JAK2-STAT5 signaling pathway, and activated STAT5 (pSTAT5) promotes milk synthesis.

As a result of the impact on serum lipids, both maternal BMI and diet are important determinants of milk FAs, lipid content, and total caloric content. The BMI directly correlates with milk fat content. Notably, elevated plasma levels of triglycerides, free FAs, and particularly, saturated long-chain FAs such as palmitic acid are evident in women with obesity during pregnancy. Palmitate is the major saturated FA in human milk, representing 20-25% of FAs. In contrast to the BMI, the dietary effects are more complex. Whereas a maternal high-fat diet may increase total milk fat as a result of long-chain FAs, a low-fat diet increases the de novo MEC synthesis production of short- and medium-chain FAs.

One embodiment of the present disclosure provides a method for improving the quality of breast milk in a female human individual that provides breast milk, or expects to provide breast milk, to a child, comprising administering to the individual an agent that increases the individual's sensitivity to insulin.

In some embodiments, the agent is selected from the group consisting of: metformin; rosiglitazone; pioglitazone; D-chiro-inositol; catechin; N-acetyl cysteine; an anti-CRFR2 antibody; and an anti-Fyn antibody. In some embodiments, the agent is metformin.

In some embodiments, the individual does not have diabetes.

Also provided, in one embodiment, is a method for improving the quality of breast milk in a female human individual that provides breast milk, or expects to provide breast milk, to a child, comprising administering to the individual an agent that:
(a) reduces the substrate uptake, synthesis or secretion of long chain fatty acids;
(b) reduces the substrate uptake, synthesis or secretion of short chain fatty acids;
(c) increases the amino acid uptake, protein synthesis or protein secretion of proteins; or
(d) reduces the uptake or synthesis of lactose.

In some embodiments, the agent:
(a) inhibits the expression or activity of lipoprotein lipase, fatty acid synthase, SREBP, PPAR or LCFA;
(b) inhibits the expression or activity of acetyl COA, hydroxybutyrate, acetyl CoA carboxylase, acylthioesterase, or MCFA;
(c) increases the expression or activity of amino acid transporter, mTOR, casein, or lactalbumin; or
(d) inhibits the expression or activity of GLUT1, UDP-galactose, lactose synthase, or α-lactalbumin.

In some embodiments, the agent is selected from the group consisting of orlistat, omeprazole, linoleic acid or conjugated linoleic acid, GW9662, CP-640186, a growth hormone, leucine, methionine, phlorizin, phloretin, theophylline, progesterone, and inhibitory RNA molecules. In some embodiments, the agent is orlistat.

In some embodiments, the individual is overweight. In some embodiments, the individual has a BMI >20, >25, or >30, >35, >40 or >50. In some embodiments, the individual is pregnant. In some embodiments, the individual has given birth within the previous 2 years. In some embodiments, the individual is breastfeeding.

In some embodiments, the agent is administered orally. In some embodiments, the agent is injected to the breast.

In some embodiments, the child is overweight. In some embodiments, the child has a BMI >20, >25, or >30, >35, >40 or >50.

In some embodiments, the agent is administered daily, once every 2 days, 3 days, 4 days, 5 days, 6 days, 7 days, 2 weeks, 3 weeks or 4 weeks.

In some embodiments, the individual is not otherwise prescribed for administration of the agent. In some embodiments, the agent is administered at a dose or frequency, or both, for when being used for treating another indication.

In some embodiments, the method comprises administering to the individual an agent that increases the individual's sensitivity to insulin and a second agent that (a) reduces the substrate uptake, synthesis or secretion of long chain fatty acids; (b) reduces the substrate uptake, synthesis or secretion of short chain fatty acids; (c) increases the amino acid uptake, protein synthesis or protein secretion of proteins; or (d) reduces the uptake or synthesis of lactose.

### SUMMARY

Despite mixed evidence that breastfeeding reduces the incidence of childhood obesity, among breastfed large-for-gestational-age infants, childhood obesity rates are nearly 50% greater than among formula-fed appropriate-for-gestational-age infants. Even among exclusively breastfed appropriate-for-gestational-age infants, a subgroup develops early-onset obesity. Among infants exposed to maternal diabetes in utero, those consuming the largest amount of human milk displayed the highest body weight at 2 years. Although the value of human milk is beyond question, it is contemplated that the variation of milk composition in women with high BMI or high-fat diets may contribute to a rapid and excessive infant weight gain. Increased infant weight at 6 at 12 months of age is a significant predictor of childhood and adult obesity. The instant inventors investigated the mechanisms by which long-chain saturated FAs modulate milk lipid production.

Utilizing a new 3-D mammary epithelial cell (MEC) primary culture model, the inventors recreated the milk production pathway in vitro and examined the effects of exogenous palmitate (conjugated to albumin) on milk triglyceride content. The inventors further determined the effects of exogenous palmitate on the protein and mRNA expression of key factors involved in milk lipid synthesis in MECs and secreted “milk” in the apical chamber. Moreover, the inventors tested whether a lipase inhibitor, orlistat, could prevent palmitate-induced milk production in MEC culture.

The important findings of palmitate-mediated increased MEC synthesis and production of “milk” triglycerides, and its prevention by a lipase inhibitor provide evidence that the 3-D mouse MEC primary culture can be used to study “milk” secretion in vitro. This has implications for modulating human milk production and composition by maternal diet or pharmacologic intervention for optimal infant nutrition and growth. Furthermore, the cellular MEC responses of mRNA activation evident in secreted “milk” suggest that there is a potential to monitor human mammary cell responses via milk mRNA content.

Another unexpected finding is that maternal insulin concentration and HOMA-IR (Homeostatic Model Assessment of Insulin Resistance) were highly correlated with first milk triglycerides. It is contemplated that patients with insulin resistance have proportionately less insulin resistance at the lactating breast as compared to liver, adipose and muscle. Thus, the relatively increased serum insulin levels in these patients would have increased local mammary epithelial cell effects, promoting cellular fatty acid uptake and incorporation into milk. Accordingly, it is believed that increased systemic insulin sensitivity (e.g., with agents such as metformin) can both reduce serum triglycerides and serum insulin, and reduce milk fatty acid contents.

### BRIEF DESCRIPTION OF THE DRAWINGS

FIG. 1 is a schematic of the culture model. The cell culture inserts maintain the cell polarity of mammary epithelial cells (MECs), which secrete milk components into the apical (upper) compartment from nutrient sources in the basolateral (lower) compartment.

FIG. 2. Milk Protein β-casein in Mice Mammary Epithelial Cultures (MEC). Immunostained images of F-actin (green) as a marker of epithelial cells and β-casein (red) as a marker of milk and merged image. Blue represents the nuclei stained with DAPI. Scale bars are 20 μm.

FIG. 3. Milk Protein β-casein and Milk Lipid in Mammary Epithelial Cell (MEC) Cultures. Panel 1 shows immunostained MECs images for DAPI, F-actin/BODIPY, adipophilin, with merged and enlarged images. Panels 2 and 3 show immunostained MECs images for DAPI, F-actin/BODIPY, β-casein, with merged and enlarged images. Scale bar 10 μm.

FIG. 4. Secreted Milk Protein β-casein in the Apical Medium. Immunoblot of β-casein in MEC “milk” (mouse 25 kDa), human milk as a positive control (23 kDa), and lactogenic medium (no band detected).

FIG. 5. Effects of Palmitate on Milk Protein β-casein and Lipid Synthesis in Mammary Epithelial Cells. MECs were treated with varying doses of palmitic acid added to the lactation medium in the basolateral chamber for 48 h. Cellular MECs were analyzed for (a) protein expression (representative immunoblot shown) and (b) mRNA expression of milk lipid regulators. Each treatment was performed in quadruplicate, and differences between treated and untreated MECs were compared using ANOVA with Dunnett's post-hoc test. Values are fold changes (Mean±SE); * p<0.001 treated vs. untreated MECs.

FIG. 6. Effects of Palmitate on Secreted Milk Protein β-casein and Milk Lipid. MECs were treated with varying doses of palmitic acid added to the lactation medium in the basolateral chamber for 48 h. The apical medium was analyzed for (a) protein expression of β-casein (representative immunoblot shown) and triglyceride concentration. (b) mRNA expression of lipid enzymes. Each treatment was performed in quadruplicate, and differences between treated and untreated MECs were compared using ANOVA with Dunnett's post-hoc test. Values arc fold changes (Mean±SE); * p<0.001 treated vs. untreated MECs.

FIG. 7. Effects of Orlistat on the Lipid Content. MECs were treated with palmitic acid and orlistat added to the lactation medium in the basolateral chamber for 48 h. MEC “milk” was analyzed for triglyceride levels. Each treatment was performed in quadruplicate, and differences between treated and untreated MECs were compared using ANOVA with Dunnett's post-hoc test. Values are Mean±SE; * p<0.01 treated vs. untreated MECs.

FIG. 8. Treatment with FITC and FITC-Dextran. MECs were treated with palmitic acid and orlistat added to the lactation medium in the basolateral chamber for 48 h. FITC or FITC-dex was added to the basolateral chamber, and the cells were incubated for 1 h; the basolateral and apical media were aspirated separately, and fluorescence intensity was measured. Values are AU for FITC and percent of flux into the apical chamber/total FITC (Mean±SE). Each treatment was performed in quadruplicate.

FIG. 9 shows that milk of women with OW/OB has markedly increased caloric content.

FIG. 10 shows that milk from both low fat (LF) and high fat (HF) women increased significantly in fat and caloric content from first to last sample, and the net increase in fat (6.3 vs 3.1 g/dL) and calorie (55 vs 25 kcal/dL) was significantly greater in HF vs LF.

FIG. 11 shows production of D- and L-lactate following control infant formula (IF) and IF plus LNnT. * p<0.05 IF vs control.

FIG. 12 shows milk fat and calorie content of eutrophic and OW/OB women. Values are Mean±SEM; Eutrophic N=9, OW/OB N=11. #OW/OB vs Eutrophic; * First vs Last milk sample.

FIG. 13 shows milk fatty acid content of eutrophic and OW/OB women. Values are Mean±SEM. Eutrophic N=9 and OW/OB N=11. #OW/OB vs Eutrophic.

FIG. 14 shows linear regression of maternal serum triglycerides vs first milk triglyceride. N=20 of combined Eutrophic and OW/OB groups.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

In certain embodiments, as used herein, the phrase “one or more” refers to one to five. In certain embodiments, as used herein, the phrase “one or more” refers to one to three.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

In many cases, the compounds of this disclosure are capable of forming acid and/or base salts by virtue of the presence of amino, and/or carboxyl groups, or groups similar thereto.

Provided are also or a pharmaceutically acceptable salt, isotopically enriched analog, deuterated analog, stereoisomer, mixture of stereoisomers, and prodrugs of the compounds described herein. “Pharmaceutically acceptable” or “physiologically acceptable” refer to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds described herein, and the like. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition. The therapeutically effective amount may vary depending on the subject, and disease or condition being treated, the weight and age of the subject, the severity of the disease or condition, and the manner of administering, which can readily be determined by one or ordinary skill in the art.

### Compositions and Methods for the Modulation of Human Breast Milk Composition

Numerous reports have confirmed that childhood obesity is a major risk factor for adult obesity and that the children of overweight/obese (OW/OB) parents have an increased risk of obesity. The underlying mechanism of programmed offspring obesity is a result of early developmentally increased appetite drive and reduced satiety with excessive weight gain during the nursing period. Contributing further to infant OW/OB is the finding that the milk of women with OW/OB is high in fat and calories. Animal studies confirmed that mice born to and nursed by obese/high-fat-diet dams demonstrated early-life and adult obesity. However, if the offspring of obese/high-fat-diet dams arc cross-fostered and nursed by control (non-obese/normal diet) dams, they grow to a normal weight as adults. Remarkably, offspring of control dams who were cross-fostered and nursed by obese/high fat diet dams also demonstrated early-life and adult obesity. These findings suggest that a compendium of factors (newborn growth trajectory, intrinsic infant appetite, breast milk nutrient, and caloric content) contribute to maintain the enhanced newborn growth. Hence, knowledge of the mechanisms of milk synthesis/production and approaches to modulate milk composition is critical to prevent excess infant weight gain.

It was discovered herein that the mouse MEC culture simulated the production of mammary gland milk production in vivo. Within epithelial cells, actin filaments are distributed predominantly at the plasma membrane and are highest in concentration at the apical membrane. MEC immunostaining for F-actin confirmed the overall intact shape and structure of the mammary epithelial cells. Furthermore, the positive staining of β-casein confirmed the presence of milk proteins. MECs further demonstrated the presence of milk lipids and intact milk fat globules in both intracellular and extracellular compartments. Neutral lipids (stained by BODIPY) mainly in the form of triglycerides are synthesized and packaged into cytoplasmic lipid droplets which are coated by the adipophilin protein. In human and mouse milk, adipophilin co-localizes with secreted milk lipid globules and is significantly associated with the milk lipid content.

Palmitate, the most common saturated FA accounting for 20-30% of total FAs in the human body, is obtained via the diet or synthesized endogenously via de novo lipogenesis. Hence, its serum concentration in patients varies depending upon diet and physiological conditions, with higher levels demonstrated in patients with increased BMI. The normal levels of adult serum triglycerides are less than 1.7 mmol/L (150 mg/dL) with normal nonesterified FA (free FA) levels approximating 10% of the triglyceride levels, of which palmitate comprises approximately 30% (˜0.06 mmol/L). The lactogenic medium contained only 0.08 mmol/L of triglyceride and 0.03 mmol/L (0.84 mg/dL) of free FA, of which 21% was palmitate (0.006 mmol/L). The experimental examples supplemented the lactogenic medium with palmitate FA at 0.05 and 0.1 mmol/L, reflecting the normal serum palmitate concentrations.

The addition of palmitate to the lactogenic medium stimulated MEC “milk” production, as evidenced by the increased casein synthesis and secretion of both casein and triglyceride. The addition of Na-palmitate at 100 μmol/L with a molecular weight of 278 g (2.78 mg/dL) doubled the secretion of MEC triglyceride, increasing “milk” concentration by ˜7 mg/dL. Importantly, the increase in milk fat was exclusively due to an increased palmitate (C16) and palmitoleate (C16:1) incorporation into milk fat.

MEC triglyceride synthesis is dependent upon the uptake of FAs from the lactation medium. The uptake of long-chain FAs from albumin-bound FAs and lipoproteins is facilitated by a membrane lipase and FA transport proteins. The enzyme LPL functions to hydrolyze circulation-derived lipids into free FAs, which are subsequently transported as free FAs into cells by CD36. Palmitate (conjugated to albumin) supplementation increased the mRNA expression of MEC Lpl (long-chain FA uptake enzyme) and Fas and the protein expression of FAS, pSTAT5, and SREBP. Prolactin/STAT5 signaling plays a central role in milk production ability in MECs. Prolactin binds to the prolactin receptor in MECs, and STAT5 is then phosphorylated through Janus-Activating Kinase 2 (JAK2). Phosphorylated STAT5 forms dimers that translocate into the nuclei to regulate the transcription of genes related to milk production. Palmitate-enriched diets cause SREBP1 activation, and SREBP1 facilitates triglyceride synthesis in MECs. Palmitate may have additional effects on MEC activity, as it exerts multiple physiological functions at the cellular level. For example, palmitate stimulates skeletal muscle glucose uptake via AMPK and Akt activation, increases CCL4 expression in monocytes, and increases the secretion of pro-inflammatory cytokines. Importantly, the increase in MEC cellular Lpl and Fas mRNA was reflected by increased mRNA in MEC “milk”.

Unexpectedly, the addition of orlistat, an LPL inhibitor, reversed the effects of palmitate stimulation on MEC “milk” triglyceride. Orlistat is used as an anti-obesity drug and acts by binding to the active sites of LPL, thus inhibiting the hydrolysis of triglycerides to free FAs. When used in humans, orlistat acts primarily within the gastrointestinal tract, as there is minimal systemic absorption. Treatment of in vitro cultures at an orlistat-to-palmitate ratio of 1:1 demonstrated cell viability using an orlistat concentration of 250 μM.

Palmitate stimulated both LPL and FAS, and treatment with orlistat suppressed MEC triglyceride production. It is contemplated that palmitate may have a stimulatory effect on LPL, with the increase in FAS representing a response to increased fatty acid substrates.

In view of the increased concentration of FAs in women with increased BMI or in response to a high-fat diet, it is likely that both diet and BMI are in part responsible for the increased fat content and caloric content of breast milk in these individuals. As increased human milk fat and caloric content may contribute to excess infant weight gain, it can be of value to reduce breast milk fat content. The use of select agents targeting maternal serum composition and MEC uptake, as well as synthesis and secretory pathways may be of benefit in modulating breast milk composition and preventing overweight/obesity in select newborns.

Another unexpected finding is that maternal insulin concentration and HOMA-IR (Homeostatic Model Assessment of Insulin Resistance) were highly correlated with first milk triglycerides. It is contemplated that patients with insulin resistance have proportionately less insulin resistance at the lactating breast as compared to liver, adipose and muscle. Thus, the relatively increased serum insulin levels in these patients would have increased local mammary epithelial cell effects, promoting cellular fatty acid uptake and incorporation into milk. Accordingly, it is believed that increased systemic insulin sensitivity (e.g., with agents such as metformin) can both reduce serum triglycerides and serum insulin, and reduce milk fatty acid contents.

### Insulin Sensitizing Agents

In accordance with one embodiment of the present disclosure, therefore, provided is a method for improving the quality of breast milk in a female human individual that provides breast milk, or expects to provide breast milk, to a child. In one embodiment, the method entails administering to the individual an agent that increases the individual's sensitivity to insulin.

Agents capable of increases an individual's sensitivity to insulin are known in the art. For instance, metformin (N,N-Dimethylimidodicarbonimidic diamide), sold under the brand name Glucophage, is able to decrease glucose production in the liver, and increase insulin sensitivity in body tissues. Metformin is a biguanide antihyperglycemic agent.

Rosiglitazone ((RS)-5-[4-(2-[methyl(pyridin-2-yl)amino]ethoxy)benzyl]thiazolidine-2,4-dione; trade name Avandia) is an antidiabetic drug in the thiazolidinedione class. It works as an insulin sensitizer, by binding to the PPAR in fat cells and making the cells more responsive to insulin.

Pioglitazone ((RS)-5-(4-[2-(5-ethylpyridin-2-yl)ethoxy]benzyl)thiazolidine-2,4-dione; sold under the brand name Actos), is in the thiazolidinedione (TZD) class and works by improving sensitivity of tissues to insulin.

D-chiro-inositol (1D-chiro-Inositol, or DCI) is a member of a family of inositol. It is known to be an important secondary messenger in insulin signal transduction and can sensitize an individual's response to insulin.

Catechin ((2R,3S)-2-(3,4-Dihydroxyphenyl)-3,4-dihydro-2H-chromene-3,5,7-triol) is a flavan-3-ol and belongs to the subgroup of polyphenols called flavonoids. It has been reported that catechin is capable of sensitizing an individual's response to insulin (see, e.g., U.S. Patent Application Publication No. US20090264519A1).

Another example agent that is capable of increasing an individual's sensitivity to insulin is N-acetyl cysteine (see, e.g., U.S. Pat. No. 6,258,848).

Agents capable of inhibiting or reducing corticotropin releasing factor 2 (CRFR2) signaling (“CRFR2 inhibitors”) have been reported to be able to increase insulin sensitivity (see, e.g., U.S. Pat. No. 7,815,905). Examples of CRFR2 inhibitors include anti-CRFR2 antibodies.

Agents capable of inhibiting or reducing Fyn kinase, or the interaction between Fyn and LKB1 (“Fyn kinase inhibitors”) have been reported to be able to increase insulin sensitivity (see, e.g., U.S. Pat. No. 7,815,905). Examples of Fyn kinase inhibitors include anti-Fyn antibodies.

In some embodiments, the individual does not have a disease or condition that requires a treatment by the agent. In some embodiments, the individual does not have diabetes, or does not have a prescription that otherwise (other than for breastfeeding) includes the agent.

### Modulation of Fat or Calorie Content of Milk

Another embodiment of the present disclosure provides a method for improving the quality of breast milk in a female human individual that provides breast milk, or expects to provide breast milk, to a child.

In some embodiments, the method entails administering to the individual an agent that reduces the substrate uptake, synthesis or secretion of long chain fatty acids. In some embodiments, such an agent inhibits the expression or activity of lipoprotein lipase, fatty acid synthase, SREBP (sterol regulatory element-binding protein), PPAR (peroxisome proliferator-activated receptor) or LCFA (long chain fatty acids).

In some embodiments, the method entails administering to the individual an agent that reduces the substrate uptake, synthesis or secretion of short chain fatty acids. In some embodiments, such an agent inhibits the expression or activity of acetyl COA, hydroxybutyrate, acetyl CoA carboxylase, acylthioesterase, or MCFA (medium-chain fatty acids).

In some embodiments, the method entails administering to the individual an agent that increases the amino acid uptake, protein synthesis or protein secretion of proteins. In some embodiments, such an agent increases the expression or activity of amino acid transporter, mTOR (mechanistic target of rapamycin), casein, or lactalbumin.

In some embodiments, the method entails administering to the individual an agent that reduces the uptake or synthesis of lactose. In some embodiments, such an agent inhibits the expression or activity of GLUT1 (glucose transporter 1), UDP-galactose, lactose synthase, or α-lactalbumin.

Types of agents and their examples are further provided in Table 1 below.

TABLE 1
Agents Modulating Milk Fat or Calorie Contents

| | Objective: | | | Action | |
|---|---|---|---|---|---|
| Breast Milk | Reduce or | | Enzyme or | (Inhibition or | Example |
| Component | Increase | Process | Target | Stimulation) | Agents |
| Fat: long chain | Reduce | Substrate | Lipoprotein | Inhibition | orlistat |
| fatty acids | | Uptake | Lipase | | |
| | | Synthesis | Fatty Acid | Inhibition | omeprazole |
| | | | Synthase | | |
| | | | SREBP | Inhibition | Conjugated |
| | | | | | Linoleic Acid |
| | | | PPAR | Inhibition | GW9662 |
| | | Secretion | LCFA | | |
| Fat: short chain | Reduce | Substrate | acetyl CoA, | Inhibition | |
| fatty acids | | Uptake | hydroxybutyrate | | |
| | | Synthesis | acetyl CoA | Inhibition | CP-640186 |
| | | | carboxylase | | |
| | | | acylthioesterase | Inhibition | |
| | | Secretion | MCFA | | |
| Protein | Increase | Amino Acid | AA Transporters | Stimulation | Growth |
| | | Uptake | | | hormone |
| | | Synthesis | mTOR | Stimulation | leucine, |
| | | | | | methionine |
| | | Secretion | casein, | Stimulation | |
| | | | lactalbumin | | |
| Carbohydrates: | Reduce | Uptake | GLUT1, UDP- | Inhibition | phlorizin, |
| Lactose | | | galactose | | phloretin |
| | | Synthesis | Lactose synthase | Inhibition | Theophylline |
| | | | α-lactalbumin | Inhibition | Progesterone |

Examples of such agents include, without limitation, orlistat, omeprazole, linoleic acid or conjugated linoleic acid, GW9662, CP-640186, a growth hormone, leucine, methionine, phlorizin, phloretin, theophylline, progesterone, and inhibitory RNA molecules. In some embodiments, the agent is orlistat.

In some embodiments, the individual is further administered an insulin sensitizing agent, such as metformin; rosiglitazone; pioglitazone; D-chiro-inositol; catechin; N-acetyl cysteine; an anti-CRFR2 antibody; and an anti-Fyn antibody.

Agents capable of inhibiting the biological activity or expression of a target protein (e.g., CRFR2, Fyn kinase, SREBP, PPAR, acetyl CoA carboxylase, acylthioesterase, GLUT1, UDP-galactose, and Lactose synthase) can be readily obtained. One such example is an antibody or antigen-binding fragment. Types of antibodies and fragments are described in more detail above, and methods of obtaining antibodies and fragments are known in the art. In one embodiment, the agent is an anti-CRFR2 antibody or antigen-binding fragment. In one embodiment, the agent is an anti-Fyn antibody or antigen-binding fragment.

Another example of such agents is an inhibitory RNA. An inhibitory RNA is an RNA molecule that can inhibit gene expression at the post-transcriptional level. There are several types of inhibitory RNA, as further set forth below.

MicroRNAs (miRNAs): miRNAs are small non-coding RNAs that regulate gene expression by targeting specific mRNAs for degradation or translational repression. miRNAs arc transcribed from DNA and then processed by the cell into mature miRNAs that can recognize and bind to complementary sequences on target mRNAs, leading to their degradation or translational repression.

Small interfering RNAs (siRNAs): siRNAs are another type of small non-coding RNA that can induce gene silencing by targeting specific mRNAs for degradation or translational repression. siRNAs are typically introduced into cells by transfection or viral transduction and can be used for research or therapeutic purposes.

Short hairpin RNAs (shRNAs): shRNAs are RNA molecules that can induce gene silencing by mimicking the structure of an miRNA precursor. They are typically introduced into cells by transfection or viral transduction and can be used for research or therapeutic purposes.

Piwi-interacting RNAs (piRNAs): piRNAs are a type of small non-coding RNA that play a role in regulating transposons and maintaining genomic stability in germ cells. piRNAs interact with a class of proteins known as Piwi proteins and can induce gene silencing by epigenetic mechanisms, such as DNA methylation or histone modification.

Anti-sense RNAs (asRNAs): asRNAs are RNA molecules that are complementary to specific mRNAs and can induce gene silencing by hybridizing to the mRNA and preventing its translation or promoting its degradation.

An agent that inhibits the biological activity or expression of a target protein, such as MARCH5, can also be a small molecule. Such small molecules can be identified readily from library screening, as exemplified in the experimental examples. Without limitation, a small molecule that inhibits MARCH5 may be pitavastatin, lonafarnib, tucidinostat, belinostat, mocetinostat, pracinostat, risedronate, entinostat, chidamide, vorinostat, or a salt thereof. In one embodiment, a small molecule that inhibits MARCH5 is pitavastatin or pitavastatin calcium.

Agents that can increase the biological activity or expression of a protein (e.g., amino acid transporter, mTOR proteins, and casein) can also be readily obtained. In one example, the agent is a recombinant version of the target protein. Alternatively, a polynucleotide, such as DNA or mRNA, that encodes the target protein can also be used. In another example, the endogenous gene of the target protein may be engineered to increase expression. Without limitation, in one embodiment, the agent is a recombinant casein protein or polynucleotide encoding the casein protein.

In some embodiments, the individual is overweight. For instance, the individual may have a BMI that is >20, >25, or >30, >35, >40 or >50.

In some embodiments, the individual is pregnant. In some embodiments, the individual is within the 2^{nd} trimester. in some embodiments, the individual is within the 3^{rd} trimester.

In some embodiments, the individual has given birth within the previous 2 years, 18 months, 12 months, 8 months, 6 months, 5 months, 4 months, 3 months, 2 months or 1 month. In some embodiments, the individual is breastfeeding.

In some embodiments, the child is overweight. In some embodiments, the child has a BMI that is >20, >25, or >30, >35, >40 or >50.

In some embodiments, the individual is not otherwise prescribed for administration of the agent. Alternatively, in some embodiments, the agent is administered at a dose or frequency, or both, lower than (e.g., <90%, 80%, 70%, 60%, 50%, 40%, 30%, or 20% for dose, or dose x frequency) for when being used for treating another indication (e.g., diabetes).

The agent may be administered orally, intramuscularly or intravenously. In some embodiments, the agent is injected to either or both of the breasts.

In some embodiments, the agent is administered daily, once every 2 days, 3 days, 4 days, 5 days, 6 days, 7 days, 2 weeks, 3 weeks or 4 weeks.

Agents provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound of Formula I may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

### EXPERIMENTAL EXAMPLES

### Example 1. Modulation of Milk and Lipid Synthesis and Secretion in a 3-Dimensional Mouse Mammary Epithelial Cell Culture Model: Effects of Palmitate and Orlistat

This example established a new 3-D mammary epithelial cell (MEC) primary culture model to recreate the milk production pathway in vitro and examined the effects of exogenous palmitate (conjugated to albumin) on milk triglyceride content. The effects of exogenous palmitate on the protein and mRNA expression of key factors involved in milk lipid synthesis was determined in MECs and secreted “milk” in the apical chamber. In addition, it was tested whether a lipase inhibitor could prevent palmitate-induced milk production in MEC culture.

### Materials and Methods

Animals: Studies were approved by the Animal Care and Use Committee of The Lundquist Institute at Harbor University of California, Los Angeles, and were in accordance with the American Association for Accreditation of Laboratory Care and National Institutes of Health guidelines. Virgin female ICR mice (10-12 weeks of age; N=6), purchased from Charles River Laboratories (San Diego, CA, USA), were housed in a facility at 22° C. with regulated humidity and a controlled 12:12 h light/dark cycle. The mice had free access to standard laboratory chow (Lab-Diet 5001, Brentwood, MO, USA) and water.

Materials: RPM1-1640 medium, protease inhibitor cocktail, BODIPY™ 493/503 (D3922), AlexaFluor™ 488 phalloidin (R37110), pSTAT5 (701063) and BAC™ Protein Assay Kit were purchased from Thermo Fisher Scientific (Irwindale, CA, USA). Trypsin, dexamethasone (mammalian D2915), epidermal growth factor (human E9644), insulin (bovine I6634), prolactin (mouse SRP4688), palmitate (sodium salt), fluorometric assay (MAK266), orlistat (O4139) and fluorescein isothiocyanate-dextran 3000 were purchased from Sigma-Aldrich (St. Louis, MO, USA).

The RIPA solution was obtained from Cell Signaling (Beverly, MA, USA), collagenase 111 from STEMCELL Technologies Inc (Cambridge, MA, USA), fetal bovine serum from Corning Life Sciences, 0.4 μm pore size cell culture inserts, from BD Biosciences (La Jolla, CA, USA) and triglyceride assay kit from Bio Vision (K622; Milpitas, CA, USA). The primary antibodies anti-FAS (SC-20140) and anti-SREBP (SC-8984) were from Santa Cruz Biotechnology, Inc (Dallas, TX), the anti-β-casein antibody from Abbexa (abx430060, Sugar Land, TX, USA), and the anti-adipophilin antibody from LSBio (LS-C146977-100, Seattle, WA, USA).

The growth medium consisted of RPMI-1640 supplemented with 10% FBS, 10 μg/mL of insulin, 10 ng/ml of EGF, 100 U/mL of penicillin, and 100 μg/mL of streptomycin. The lactogenic medium consisted of growth medium plus 0.5 U/mL of prolactin and 1 μM dexamethasone. The palmitate-albumin conjugate stock solution contained a palmitate solution (2 mM/L in 50% ethanol) and FA-free BSA (2%) and was diluted in lactation medium to provide 50 and 100 μmol/L palmitate concentrations. Orlistat was dissolved in ethanol.

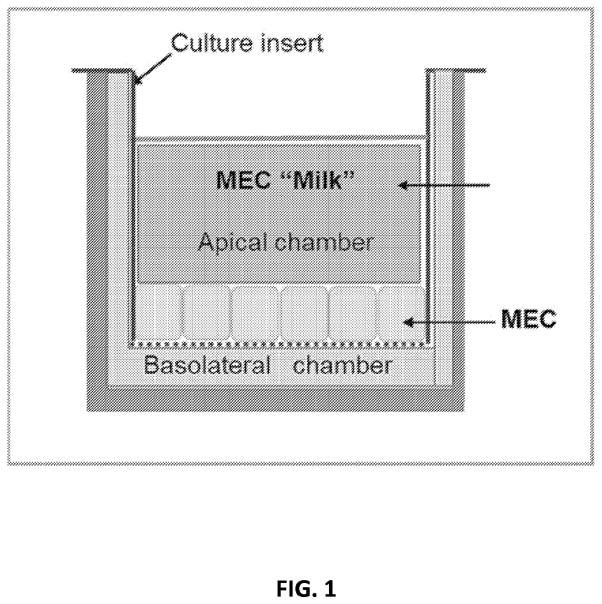

3-D Cell Culture Model: This example recreated the milk production pathway in mammary epithelial cell (MEC) primary cultures. The method uses cell culture inserts to maintain the cell polarity of MECs, which secrete milk components into the apical (upper) compartment from nutrient sources in the basolateral (lower) compartment (FIG. 1). Briefly, the mice (N=6) were euthanized, and the 4th abdominal mammary glands from both sides were collected in PBS buffer for the isolation of MECs. The mammary glands were minced and incubated in RPMI-1640 medium containing collagenase III (1.5 mg/mL) for 2 h at 37° C. with gentle shaking, followed by centrifugation (600×g for 1 min). The pellet was re-suspended in RPMI-1640 with 0.2% trypsin for 5 min at room temperature. The fragments of the mammary epithelium were dissociated by gentle pipetting with a Pasteur pipette and centrifuged (600×g for 1 min), and the pellet was re-suspended in 60% FBS (fetal bovine serum) followed by centrifugation (10×g for 1 min). The trypsin treatment and centrifugation with FBS were repeated for the isolation of mammary epithelial fragments without contaminating cells, such as fibroblasts, adipocytes, and myoepithelial cells. The epithelial fragments (0.1 mg) were subsequently seeded in 24-well cell culture plates with inserts to separate the basolateral chamber containing the lactogenic medium (nutrients) and the apical chamber containing the secreted MEC “milk”. The cells were cultured for 6 days at 37° C. in growth medium placed in the basolateral chamber. To induce lactogenesis, MECs were cultured in lactogenic medium placed in the basolateral and apical chambers for 2 days at 37° C. To induce a high milk production ability, MECs were cultured at 39° C. with the lactogenic medium in the basolateral chamber and with RMPI-1640 in the apical chamber (FIG. 1).

Treatments: Palmitate (50, 100 μmol/L) or the lipase inhibitor orlistat (100 μmol/L) together with palmitate (100 μmol/L) were added to the lactogenic medium within the basolateral chamber, and RPMI-1640 was maintained in the apical chamber. After 48 h, MECs and apical medium were collected separately for analysis, as described below.

Sample Analysis: Henceforth, we refer to cellular MECs as “MECs” and to apical medium as MEC “milk”. MECs were fixed for immunostaining or stored at −80° C. for protein and mRNA expression studies. MEC “milk” was frozen at −80° C. and analyzed for triglyceride concentration and protein and mRNA expression.

Immunostaining: MECs were fixed with 1% formaldehyde in PBS at 4° C. and then immunostained for markers of epithelial cells (F-actin), milk (β-casein), lipids (BODIPY), milk lipid droplets (adipophilin), and nuclei (DAPI). PBS containing 5% bovine serum albumin was used to block nonspecific binding. Images of MECs were acquired using a fluorescence microscope. F-actin staining (AlexaFluor™ 488 phalloidin, R37110) was performed.

Western Blot: The cells were harvested and dissolved in RIPA solution with a protease inhibitor cocktail and sonicated, and the cell lysates were processed for the analysis of protein concentration by the BAC™ Protein Assay Kit. For the detection of pSTAT5, the protein phosphatase inhibitor NaF (50 mM) was added to all buffers. MEC protein expression of β-casein (25 kDa), FAS (270 KDa), SREBP1 (68 KDa), and pSTAT5 (92 KDa) was analyzed.

MEC “milk” was similarly analyzed for protein expression of β-casein.

RT-PCR: The mRNA abundance of the target genes (lipoprotein lipase, LPL; FAS) and the reference gene (18S) in MEC and MEC “milk” was determined by RT-PCR. Briefly, RNA was extracted following the manufacturer's instructions, using the RNeasy Mini Kit (Qiagen, Valencia, CA, USA). Briefly, a guanidine-thiocyanate-containing lysis buffer and ethanol were added to the samples to promote the selective binding of RNA to the RNeasy membrane. The samples were then applied to the RNeasy Min spin column where RNA binding to the silica membrane enabled contaminants to be efficiently washed away. The purified RNA was then eluted in RNase-free water. The purity of RNA (A260/A280) for all samples was 1.8˜2.0 as determined by spectrophotometry, indicating that the samples were pure and clean. PCR was performed in 96-well optical reaction plates (Applied Biosystems, Foster City, California) on cDNA equivalent to 0.5 μg of RNA in a volume of 20 μL, using Takara PrimeScript RT master mix (RR036A, TaKaRa Bio) at 37° C. for 15 min.

PCR (TaKaRa TB Green Premix Ex Taq, RR420) was performed for the genes of interest and the 18S gene, using the ABI-Prism 7700 Sequence System (Applied Biosystems) at the following conditions: 30 s at 95° C. for 1 cycle and 5 s at 95° C., 30 s at 60° C. for 40 cycles. All samples were run in triplicate. In the control PCR, we replaced cDNA with water obtaining a threshold level (CT) value of 40, indicating no detectable PCR product under these cycle conditions. The ABI Sequence Detection System 1.6 software (Applied Biosystems) was used to select a threshold level of fluorescence that was in the linear phase of the PCR product accumulation.

The results from the RT-PCR assay were calculated as the difference between the CT for a specific mRNA gene and the CT for the reference 18S mRNA and expressed as fold change, using the formula 2^{-(ΔΔCT)}.

Triglyceride: MEC “Milk” concentration was analyzed using a fluorometric assay (MAK266, Sigma, St. Louis, MO, USA).

Cell Permeability: To assess whether mammary epithelial permeability was altered by the treatments, we measured the unidirectional flux of fluorescein isothiocyanate (FITC, molecular weight 376) and FITC-labeled dextran 3000 (FITC-dex) from the basolateral to the apical compartments. MECs were prepared as stated above and treated with palmitate and orlistat as described above. After 2 days, FITC and FITC-dex (0.5 mg/mL) were added to the basolateral chamber, and the cells were incubated for 1 h. Thereafter, the basolateral and apical media were aspirated separately, and fluorescence intensity was measured at 490/520 nm excitation/emission maxima. The permeability of the cells is expressed as percent of flux into the apical chamber/total FITC administered into the basolateral chamber.

Statistics: Mammary glands were pooled from N=6 mice for MEC cultures. Each treatment was performed in quadruplicate. NCSS statistical software was used for data analysis. The differences between treated and untreated MECs were compared using ANOVA with Dunnett's post-hoc test.

### Results

### MEC Model and Synthesis of Milk

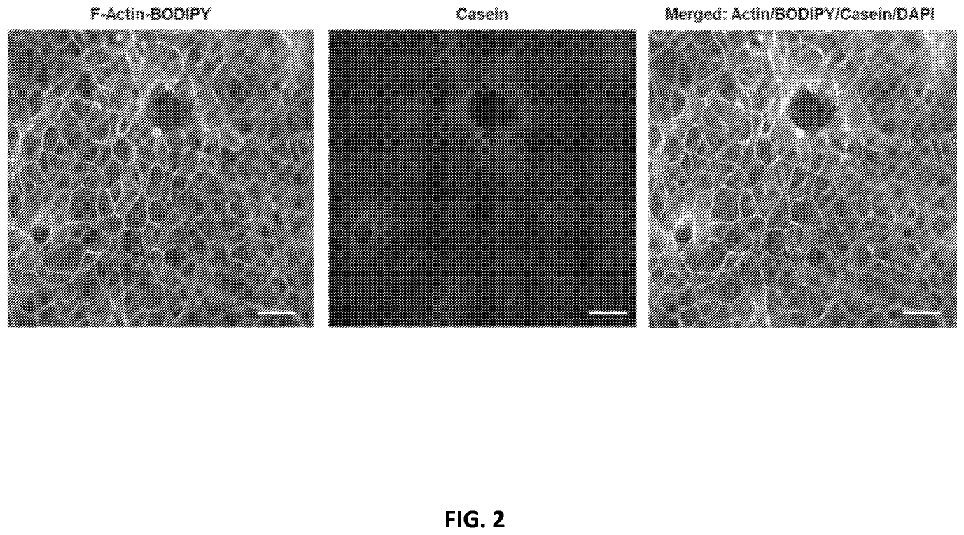

The culture model was evaluated by DAPI nuclear staining and F-actin, which confirmed the overall intact shape and structure of the mammary epithelial cells. The DAPI staining demonstrated intact MEC nuclei after lactogenic stimulation, while the staining of actin and β-casein (major milk-specific protein) showed a cytoplasmic localization of the latter, surrounding the nuclei, and confirmed the presence of milk as seen in the merged image (FIG. 2).

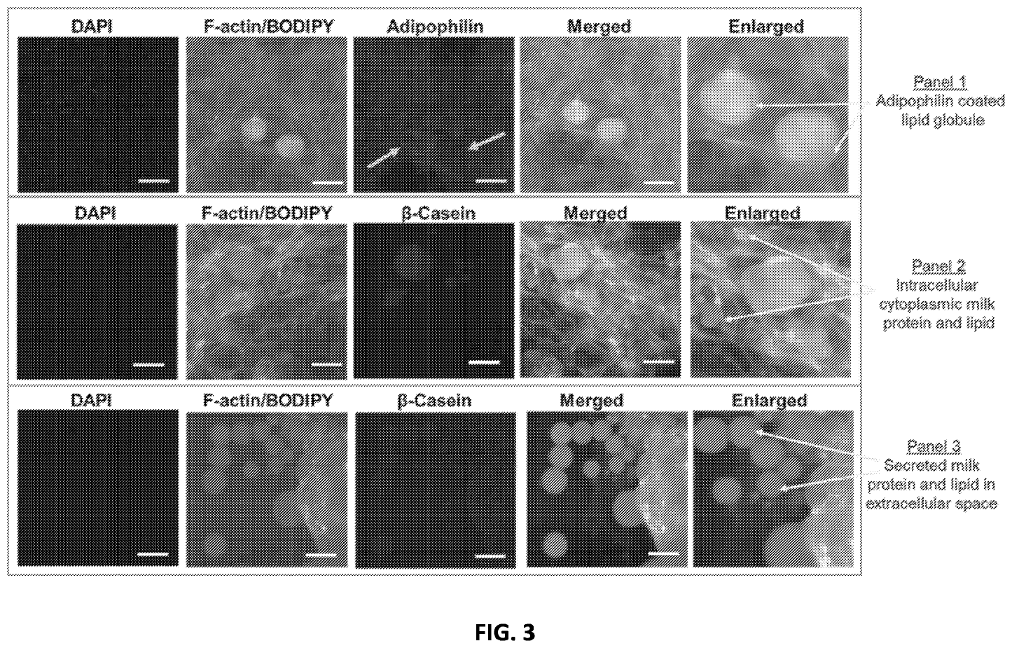

BODIPY (a marker of neutral lipids such as triglycerides) demonstrated the presence of intracellular lipid, while co-staining with adipophilin (surface protein that coats the milk lipid globules) confirmed the presence of milk globules (FIG. 3, Panel 1). Co-staining of the milk globules with casein confirmed the co-localization of milk protein and lipid in the globules and demonstrated their cytoplasmic localization (FIG. 3, Panel 2). Additional staining of β-casein together with F-actin and BODIPY confirmed the secretion of milk protein and lipid in the extracellular space of in vitro-cultured MECs (FIG. 3, Panel 3).

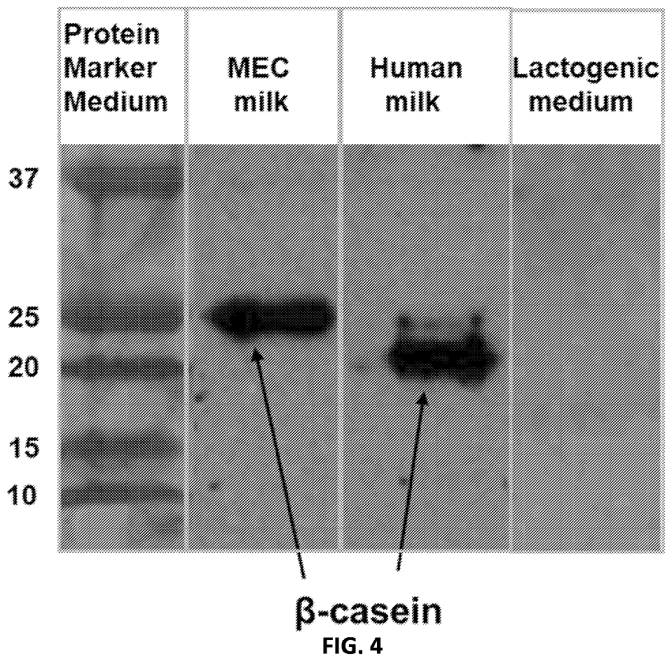

The secretion of “milk” in the culture model was further verified using Western blot for the detection of β-casein in the apical medium. Consistent with the positive control (human milk β-casein, 23 kDa), mouse β-casein in the apical medium was detected at 25 kDa (FIG. 4). No β-casein was detected in the lactogenic medium.

### Provision of Palmitate Increased Milk and Lipid Synthesis

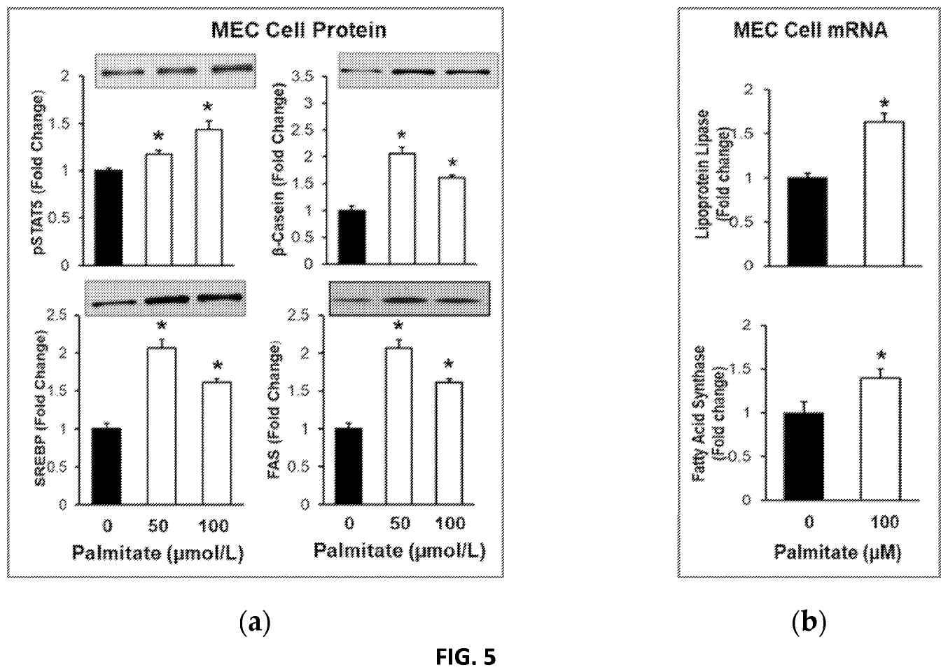

The effects of an exogenous palmitate-albumin conjugate on the protein and mRNA expression of key factors involved in milk lipid synthesis in MECs and secreted “milk” in the apical chamber were studied. Following the addition of the palmitate-albumin conjugate to the lactogenic medium, MECs demonstrated increased milk and FA synthesis, as evident by the increased protein expression of the lactogenic transcription factor pSTAT5, the milk protein β-casein, the lipogenic transcription factor SREBP1, and the lipogenic enzyme FAS. Consistent with the protein expression, the mRNA expression showed similar changes, with increased mRNA expression of the long-chain-fatty-acid-uptake enzymes Lpl and Fas (FIG. 5).

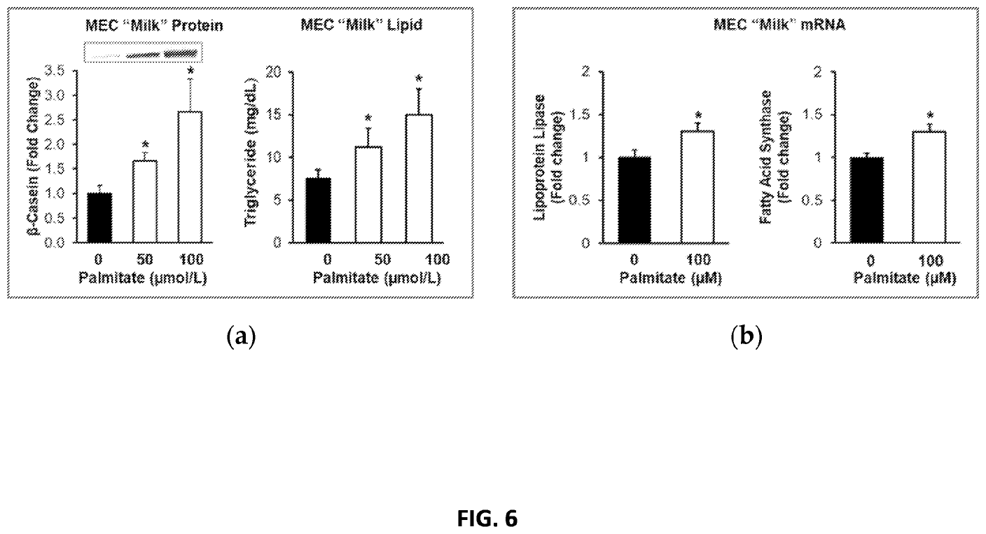

The analysis of MEC “milk” showed that with an increasing dose of palmitate, there was a dose-dependent increase in β-casein protein expression and triglyceride concentration. Notably, MEC “milk” mRNA expression of Lpl and Fas was also increased (FIG. 6).

Treatment with Orlistat

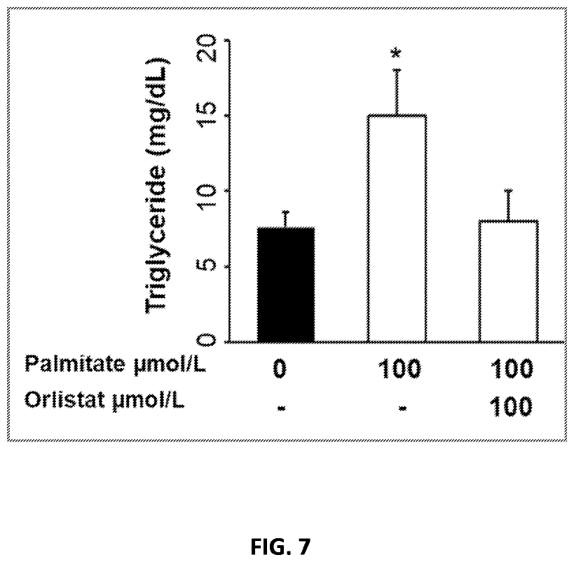

We tested whether a lipase inhibitor prevented palmitate-induced milk production in the MEC culture. Orlistat treatment normalized the palmitate-induced MEC “milk” triglyceride concentrations to that of untreated MECs (FIG. 7).

### Treatments and Cell Permeability

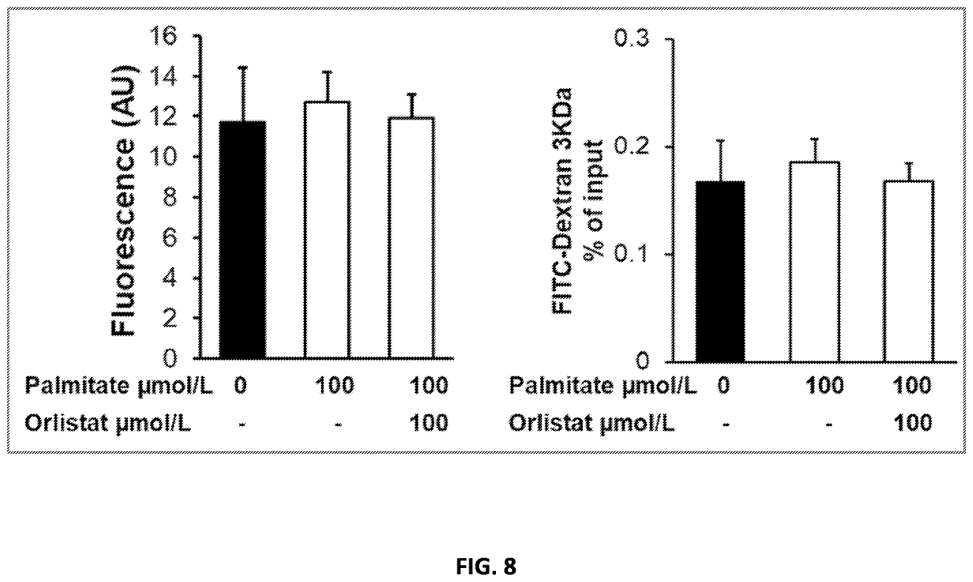

To evaluate the effect of the palmitate and orlistat treatments on epithelial barrier function, cell permeability was evaluated by measuring the unidirectional paracellular fluorescein flux and the leakage of FITC-dextran from the basolateral to the apical compartment. The fluorescence measured in the apical chamber was comparable between non-treated and treated groups. Additionally, the percent of flux of FITC-dextran from the basal to the apical chamber was overall minimal (<0.2%) and comparable between the groups, suggesting that the epithelial barrier function was not altered by the treatments (FIG. 8).

### Example 2. Human Milk Contribute to Generational Obesity

This example evaluated whether breast milk of mothers with OW/OB has higher caloric content which may promote excessive infant weight gain.

Methods: Women who delivered singleton, term neonates and were exclusive breastfeeding were studied at 7-8 weeks postpartum. Women were grouped by BMI categories (normal 18.5-24.9; OW/OB ≥25). Continuous breast milk samples were obtained from foremilk to hindmilk in 10 ml aliquots until the breast was emptied. Infant feeds were performed on the alternate breast and volume determined by infant weight change. Milk composition was analyzed (fat, total and true protein, carbohydrate, calorie; Miris) and compared between normal (N=5) and OW/OB (N=17) women, and between first and last sample using ANOVA.

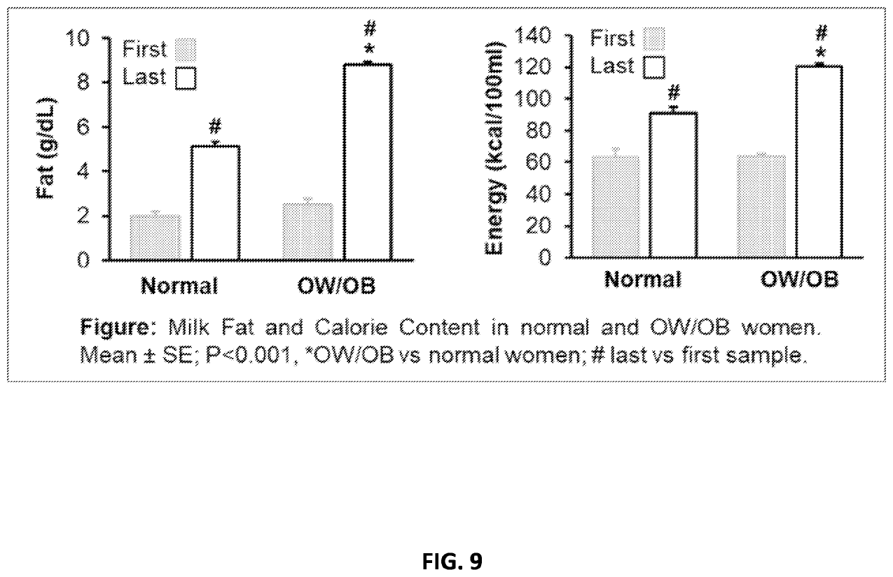

Results: Among the 22 women, the average age was 30 years and gravidity 2. Increased milk yield was obtained from women with OW/OB vs normal weight (72.9±7.5 vs 48.5±10.3 ml; P<0.05). The fat/calories in the foremilk were similar, though the hindmilk of OW/OB had significantly higher fat and caloric content (121±3 vs 90±4 kcal/dL; FIG. 9). The average milk caloric content was significantly greater among mothers with OW/OB vs normal weight (75±9 vs 114±7 kcal/dl).

Milk fat increased ˜3-fold from foremilk to hindmilk. Although human milk is optimal for infant feeding, the milk of women with OW/OB has markedly increased caloric content. Novel approaches to calibrate the caloric intake of infants of OW/OB mothers and prevent childhood obesity are needed.

### Example 3. High Vs Low Calorie Human Milk: A Cause for Overweight Infants

This example sought to determine if lactating mothers may be differentiated by the production of high and low milk fat content.

Methods: Women (N=22) who delivered singleton, term neonates and were exclusive breastfeeding were studied at 7-8 weeks postpartum. After at least 1½ hour from a prior infant feed, continuous breast milk samples were obtained from foremilk to hindmilk of the alternate breast in 10 ml aliquots until the breast was emptied. Milk samples were analyzed for composition (fat, total and true protein, carbohydrate, calorie; Miris). Milk fat categories were determined by first sample (foremilk) fat concentration.

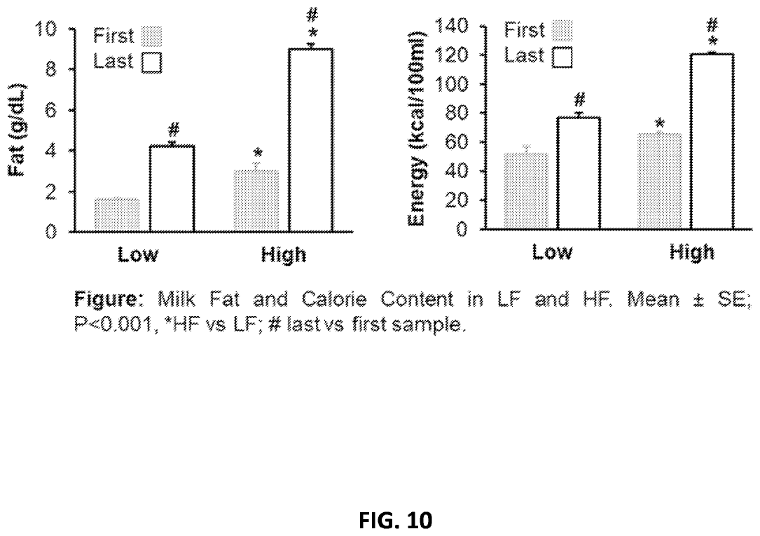

Results: The average age of women was 30 years, gravidity 2. Amongst all women, there were two distinct group based upon the first milk sample: Low Fat (LF <2.0 mg/dl; N=6) vs High Fat (≥2.0 mg/dl; N=15). Although milk from both LF and HF women increased significantly in fat and caloric content from first to last sample, the net increase in fat (6.3 vs 3.1 g/dL) and calorie (55 vs 25 kcal/dL) was significantly greater in HF vs LF (FIG. 10). Infants of women with HF vs LF had a trend towards higher birth weight (3.5 vs 3.2 kg) and the amount of milk ingested at the study feed (57 vs 41 g). Maternal BMI did not correlate with identification as LF or HF.

There is marked inter-individual variability in human milk fat and caloric content from foremilk to hindmilk. As BMI did not correlate with foremilk fat content, it is contemplated that other factors, including maternal diet, contribute to high milk fat and calorie content. The increased feed volume of higher calorie milk may promote excessive infant weight gain.

### Example 4. Insulin Resistance Associated with High Calorie and Fat Content in Milk

This example examined the entire content of the breast, from foremilk to hindmilk, in eutrophic and OW/OB women, while controlling for time of day and weeks postpartum. The results suggest that the breast milk fat content of OW/OB women may have significant caloric implications for infant growth and body composition. The association of milk calories and fat content with maternal serum lipids and hormones suggests opportunities for modulating milk composition for infants at risk of OW/OB.

### Methods

Study Participants: The study was approved by the Institutional Review Board at the Lundquist Institute at Harbor-UCLA Medical Center. This example enrolled women delivering singleton, term pregnancies after written human subjects consent was achieved. Exclusions include women with breast implants, prior breast surgery, flat/inverted nipples, tongue-tie or low birth weight infants (<2500 g), or pregestational diabetes. Women were selected for BMI (based on pre-pregnancy reported weight) 18-24.9 (eutrophic) and >25 (overweight/obese). All mothers were committed to exclusive breast feeding for at least two months. Studies were performed at 7-9 weeks postpartum (mature milk).

Women were consented for enrollment and interviewed by staff for demographic information, including but not limited to: maternal age, ethnicity, and pre-pregnancy height and weight. Additional demographic data was obtained from the electronic medical record or patient interview, including but not limited to: Height and weight at last prenatal visit, gestational age at delivery, pregnancy complications (including gestational diabetes), medications, mode of delivery, and baby's height and weight at birth and discharge and gender.

All studies were performed in the outpatient clinic of the Lundquist/Harbor-UCLA Clinical and Translational Research Center (CTRC) between 10 am and 12 μm to avoid potential circadian changes in breast milk composition. Mothers were fasted for ≥1 hour prior to study. Prior to initiation of nursing, maternal blood samples were drawn for analysis.

Breast milk samples were obtained at least ≥1.5 hours from the prior infant feed, which was from only one breast. The opposite breast was used for breast milk sampling. The nipple and areola were wiped clean and an electrical pump (Medela, IL) was applied to the breast. Continuous breast milk samples were obtained in 10 ml aliquots, and pumping continued until primary breast was emptied or there was no further milk production. Milk samples were analyzed by Miris (Uppsala, Sweden) milk analyzer and remaining samples were frozen.

Following the pumping, the infant was weighed to the nearest 1 g (Seca 757 electronic baby scale). The infant was then allowed to feed (first from the breast not used for milk sampling) and then reweighed to determine milk intake during a single feed. Additionally, supine length was measured to the nearest 0.1 cm (Infantometer SECA 416) and infant ponderal index calculated.

Blood Analysis: Serum samples were sent to Quest Diagnostics for analysis of lipid panel (triglycerides, total cholesterol, LDL-Cholesterol, HDL-Cholesterol, non-HDL Cholesterol, Cholesterol/HDL Ratio), glucose and insulin. Invitrogen (Waltham, MA, USA) ELISA kits were used to analyze plasma leptin (KAC2281) and adiponectin levels (KHP0041).

Milk Analysis: Fresh milk samples were analyzed for fat, protein and carbohydrate content using ‘Miris Human Milk Analyzer’. All samples were kept at 40° C. (Miris Heater), homogenized for 1.5 s/1 mL (Miris Ultrasonic Processor) and samples injected into the flow cell and measured in triplicate. The analyzer, which has been validated for accuracy, is a semi-solid mid-infrared (MIR) transmission spectroscopy that uses specific wavebands for determination of functional carbonyl groups (5.7 μm) for fat, amide groups (6.5 μm) for protein and hydroxyl groups (9.6 μm) for carbohydrate. True protein is corrected for non-protein-nitrogen (crude protein multiplied by 0.8), and total carbohydrate content includes lactose and oligosaccharides. Total solids are measured by drying-oven and the analyzer provides a calculation of energy using conversion factors of 4.0, 9.25, 4.4 kcal per 100 mL for carbohydrate, fat and protein, respectively.

Lipid Analysis: Lipid studies were performed at UCLA Lipidomics Core. Plasma (25 μl) and milk (25 μl) samples were extracted using a modified method described by Bligh and Dyer. Prior to biphasic extraction, an internal standard mixture consisting of 70 lipid standards across 17 subclasses was added to each sample (AB Sciex 5040156, Avanti 330827, Avanti 330830, Avanti 330828, Avanti 791642). Following two successive extractions, pooled organic layers were dried in a Thermo SpeedVac SPD300DDA using ramp setting 4 at 35° C. for 45 minutes with a total run time of 90 minutes. Lipid samples were resuspended in 1:1 methanol/dichloromethane with 10 mM ammonium acetate and transferred to robovials (Thermo 10800107) for analysis.

Samples were analyzed on the Sciex 5500 with DMS device (Lipidyzer Platform) with an expanded targeted acquisition list consisting of 1450 lipid species across 17 subclasses. Differential Mobility Device on Lipidyzer was tuned with EquiSPLASH LIPIDOMIX (Avanti 330731). Data analysis was performed on an in-house data analysis platform comparable to the Lipidyzer Workflow Manager. Detailed instrument method including settings, tuning protocol, and MRM (multiple reaction monitoring) list was as previously described. Quantitative plasma and milk values were normalized to volume (mLs) and concentrations reported as nmoles/mL.

Statistics: N=9 Eutrophic and N=11 OW/OB mother-infant were enrolled. For lipidomics, N=8 Eutrophic and N=11 samples were analyzed. Differences between maternal BMI groups and lipidomics data were analyzed by non-parametric Mann-Whitney test. Infant characteristics were analyzed by unpaired t-test. Milk data (first, middle, last) were analyzed by repeated measures of ANOVA. Values are expressed as means±SEM. Linear regression was used to test if maternal serum triglycerides and insulin significantly predicted milk triglyceride content.

### Results

Maternal Characteristics: A total of 20 women were enrolled in the study, of which 9 were of eutrophic BMI (age 29.8±2.6 years, BMI 21.2±0.6) and 11 were OW/OB (age 29±1.9 years, BMI 32.5±1.7). Subjects demographics included Asian (4), Hispanic (11), Black (4) and White, non-Hispanic (1). Infant gender was 9 male and 11 female. Among the serum values, leptin was significantly increased in OW/OB subjects. Notably, serum triglyceride (TG) levels were similar in eutrophic and OW/OB subjects, though insulin and HOMA-IR showed an increased trend.

Milk Composition: The total pumped milk volume (i.e., breast volume) was similar in the two groups (Eutrophic BMI 80±12, OW/OB 88±7 ml). Milk composition was examined in the first, the 4^{th}, 6^{th} and the last 10 ml sample from the pumped breast. Crude and total protein, carbohydrate and solids concentrations were similar between eutrophic and OW/OB women. However milk fat and caloric content were significantly increased in OW/OB women ((Repeated ANOVA Fat: F (1, 2)=7.78, p<0.01; Calorie: F (1, 2)=8.39, p<0.005)) with post hoc analysis demonstrating significant differences between BMI groups and in the first and last sample. Notably, when averaged over the four samples, caloric content levels values were 13% greater in OW/OB women.

In both eutrophic and OW/OB subjects, from the first to last sample, milk fat concentration increased 3 to 4 fold, with mid samples representing intermediate values. Caloric content similarly increased nearly 2 fold from first to last sample. Crude and true protein, carbohydrate and solid content remained unchanged from first to last sample.

Lipid Analysis: Milk lipid analysis demonstrated significantly increased milk TG from first to last sample among both Eutrophic and OW/OB subjects with a trend toward increased TG in OW/OB (P<0.07). As compared to first milk sample, the last sample demonstrated increased concentrations of free fatty acids (FFA), neutral lipid (diacylglycerols, DG), ceramide (Cer d18:1), and phospholipids (phosphatidylcholine, PC; phosphatidylethanolamine, PE; phosphatidylinositol, PI; lysophosphatidylcholine, LPC; Lysophosphatidylethanolamine, LPE; Sphingomyelin, SM). In all cases, OW/OB showed an increased trend though only Cer d18:1 and LPC were significantly higher in first milk as compared to Eutrophic subjects. In contrast, maternal plasma showed no differences except for FFA which was significantly decreased in OW/OB versus Eutrophic subjects.

Amongst the FFAs, long chain fatty acids (≥16) were significantly higher in first milk from OW/OB with a trend towards increased levels (P<0.08) in last milk sample as compared to milk from Eutrophic women. No differences were seen in C14 to C16 fatty acids.

Maternal Serum Factors and Milk Triglycerides: Linear regression analysis of maternal serum TG versus milk TG was performed to assess the association of first and last milk sample TG with serum factors. Maternal serum TG was correlated with first milk TG (R²=0.306, F (1, 18), P<0.01) but not last milk TG (R²=0.111, P<0.115). Importantly, maternal insulin concentration (R²=0.572, F (1, 18), P<0.0001) and HOMA-IR (R2=0.477, F (1, 18), P<0.0007) were highly correlated with first milk TG but not last milk TG.

Low versus High Milk Fat: Subjects were further divided into two groups (Low Fat, High Fat) based upon the fat content of the first milk sample resulting in 14 and 6 patients in the Low Fat (Fat <2 g/dL) and High Fat (Fat ≥2 g/dL) groups, respectively. In subjects with higher milk fat content, serum triglycerides (Quest Diagnostics) and plasma triglycerides (lipidomics) and insulin levels including HOMA-IR were significantly higher and results were irrespective of their BMI status.

Infant Characteristics: Infants of OW/OB mothers demonstrated a trend toward increased birth weight and increased ponderal index, with male infants of OW/OB mothers having a greater ponderal index than males of eutrophic mothers (2.80±0.05 vs 2.42±0.08, P<0.05). As described above, following the pumping of the study breast, infant feeding studies were performed on the alternate breast. Despite similar body weight at study (OW/OB: 5125±212; Eutrophic: 5290±185), the milk intake of infants of OW/OB mothers was markedly greater than that of eutrophic mothers, though there was no difference between males and females within each group.

On average, human milk contains 0.8-0.9% protein, 3-5% fat, 6.9-7.2% carbohydrates, and 0.2% minerals. Median total caloric content is 66 kcal/100 ml with an interquartile range of 62.0 to 72.5 kcal/100 ml, indicative of individual variance. Although there are few studies, previous results suggest that maternal BMI appears to be correlated with milk caloric content. Most prior studies examined a single timed or random breast milk sample. The results of the present study confirm this finding, though demonstrating important differences in milk composition from foremilk to hindmilk.

Although familial lifestyle and environmental factors may contribute to the obesity epidemic, we propose that developmental programming effects of the maternal environment during both in utero and newborn periods result in a predisposition to offspring OW/OB. In this relatively small cohort, infant birth weight demonstrated an increased trend in OW/OB subjects, consistent with data from large cohorts. Although we examined only a single feeding episode, our results indicated a nearly 40% greater milk intake among both male and female infants of OW/OB as compared to eutrophic mothers. Infant weighing before and after feeding has an estimated accuracy of ˜10% for the determination of milk intake. Whether the single increased milk intake can be extrapolated to daily milk intake can only be determined with more extensive study. However, these results are consistent with animal studies from our laboratory and others demonstrating programmed hyperphagia and increased food intake among rodent offspring from OW/OB dams.

OW/OB women had a slightly, but not significantly increased volume of breast milk obtained by pumping. There have been numerous reports of challenges of breastfeeding among OW/OB women, a result of body habitus and breast size as well as infant positioning and latching. Although only a single sample, these results suggest that perceived difficulties in OW/OB breastfeeding may not be a consequence of reduced milk volume.

Breast milk of OW/OB women demonstrated significantly increased fat and caloric content. Surprisingly, there were no differences in serum lipids between eutrophic and OW/OB subjects with OW/OB serum cholesterol trending lower than eutrophic levels. Due to maternal life style limitations with infant care and early morning infant feeds, we did not collect blood samples after overnight fasting. Most, but not all studies have demonstrated increased serum triglycerides and cholesterol in OW/OB nonpregnant and postpartum women, though lactation appears to reduce serum lipid levels. The lack of difference in serum lipids potentially may reflect effects of morning food intake prior to the study.

Despite the absence of scrum lipid differences, OW/OB women demonstrated significantly increased fat content from first to last breastmilk sample. Triglycerides (TGs) make up 98% of milk lipid content and contribute 40-50% of human milk calories. Human milk TGs result from three sources: endogenous fat stores, dietary lipids, and de novo mammary epithelial cell (MEC) synthesis. Endogenous fat stores or dietary lipids modulate serum lipids and thus contribute to long chain fatty acids (FAs) as human MECs have limited ability to synthesize C18 FAs. These long chain FA which contribute ˜80% of the total milk fat content primarily are carried by very low-density lipoproteins and are taken up by MECs following hydrolysis by MEC lipoprotein lipase. MEC de novo synthesis contributes short/medium chain FAs (C6-C14) with acetate as the primary carbon source. Whereas a maternal high fat diet may increase total milk fat as a result of long chain FAs, a low-fat diet increases MEC synthesis of short and medium chain FAs.

Maternal diet impacts milk composition, though with a delay of ˜24-36 hours. Although it is possible that maternal scrum TG differences existed throughout the day prior to the milk sampling, the present results suggest that additional factors other than BMI may regulate milk fat composition. When subjects were divided into those with Low or High fat first milk sample, the results suggest a high correlation with serum insulin and HOMA-IR levels. Linear regression analysis further supports the strong association of first milk sample with serum insulin levels and a less significant association with serum TG levels. Thus, insulin resistance, consistent with the association of first milk TG with HOMA-IR values, may be etiologic in first milk TG content. Evidence suggests that insulin resistance occur unequally among primary insulin-target sites. It is contemplated that patients with insulin resistance have proportionately less insulin resistance at the lactating breast as compared to liver, adipose and muscle. Thus, the relatively increased serum insulin levels in these patients would have increased local mammary epithelial cell effects, promoting cellular fatty acid uptake and incorporation into milk. Of note, neither serum insulin nor serum TG levels were significantly predictive of last milk TG levels, although OW/OB subjects' milk TG levels were markedly greater than eutrophic women. Together, these findings indicate that the regulatory mechanisms of foremilk and hindmilk composition differ.

With increased milk fat and caloric content, it is apparent that infants of OW/OB mothers may ingest greater caloric content at each feeding. The greater volume of feed further promotes a greater ingestion of the high-fat, high-calorie hindmilk. These findings may contribute to increased weight gain observed in infants of OW/OB mothers.

### Example 5. Determine the Impact of Altered Milk Lactose, Fatty Acid and HMO Composition on the Infant Microbiome in the In Vitro Proximal Colon Model

Maternal milk composition impacts fetal biology directly via nutrient content and indirectly via the infant microbiome, which itself has infectious, immunologic, metabolic, growth and satiety effects on infant health. This example will explore the pathways of potential modification of milk composition (lactose, FAs, HMOs) in both MEC culture and human milk, which are likely to impact the microbiome diversity and specificity.

It is contemplated that FAs and HMOs (type I and II) which are preferentially metabolized by beneficial microbiota (e.g., bifidobacteria) may alter the infant microbiome, reducing the growth of potentially pathogenic bacteria. This example will examine the impact on the infant microbiome in vitro, prior to clinical studies, using a validated dynamic proximal colon model (TIM-2). These preclinical studies will provide critical information as to potential beneficial or adverse microbiome effects, which will serve as the basis for future human intervention studies.

Preliminary Data on Effect of HMOs on infant microbiome: These in vitro studies were performed with TIM-2 107-121 inoculated with feces from 3-month old breast-fed infants (pooled microbiota of 5 infants).

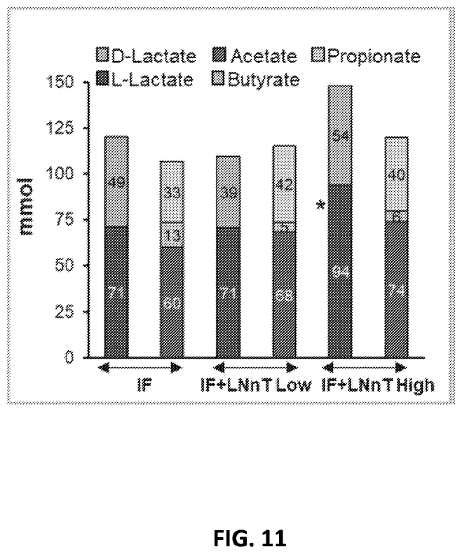

Effect of HMO lacto-N-neotetraose (LNnT) on microbiome metabolic activity: We studied the effect of the most abundant HMOs LNnT (200 and 1000 mg/L) on the metabolic activity of the gut microbiota in TIM-2 when exposed to infant formula. Analysis of microbial metabolites (D- and L-lactate) demonstrated increased L-lactate with high dose LNnT, likely due to the stimulation of bifidobacteria species that primarily produce L-lactate. These findings suggest that LNnT supplementation may of benefit for both formula and milk-fed infants. (FIG. 11).

Metabolism of 6′-SL reflects microbiota function: We studied the metabolism of 6′-SL using a 13C-label, where the lactose- and pyruvate-moiety were labeled and the ManNAc-moiety remained unlabeled. Fermentation in TIM-2 infant microbiota was followed using NMR and stable isotope-label identification. The fermentation patterns of 6′-SL indicated cross-feeding between microbes based on the appearance of isotopomers. For instance, the presence of fully-labeled lactate (m+3) directly stems from 6′-SL. The production of metabolites was correlated to the presence of bacterial taxa.

Antibiotic-induced infant microbiota alterations: Antibiotic treatment may adversely alter the infant gut microbiome. We determined the change in TIM-2 infant gut microbiota composition induced by antibiotic treatment with amoxicillin/clavulanate (A/C). The major taxa present prior to A/C were Bifidobacterium and Enterobacteriaceae. A/C markedly shifted the infant gut microbiota over time with an increase in Enterococcus and the Enterobacteriaceae family, while Veilonella and Bacteroides were reduced. At later time-points the growth of Megasphaera, Collinsella and Lachnoclostridium were inhibited. These findings demonstrate the value of TIM-2 preclinical studies to assess the impact of HMOs and other factors (e.g., antibiotics) on the infant microbiota.

TIM-2 in vitro Colon Model: TIM-2 simulates the colon and consists of four interconnected compartments containing flexible membranes in which peristaltic movements are mimicked. Conditions of healthy human infants are simulated including: body temperature, lumen pH, composition and rate of secretion fluids, delivery of a predigested substrate from the ‘ileum’, mixing and transport of intestinal contents by peristalsis, absorption of water and microbial metabolites through a dialysis system, and presence of a complex, high density, metabolically active and anaerobic microbiota of healthy human infants. The dialysis system prevents accumulation of microbial metabolites, which would otherwise inhibit or kill gut microbiota. The model will be inoculated with a pooled microbiota of healthy human infants (˜2 months) using feces collected (diaper samples) from 10 volunteers and pooled in an anaerobic cabinet to allow for a standardized microbiota. Pooling the microbiota from different individuals leads to a pool with the same metabolic capacity as observed in the individual samples. 107 The pooled microbiota will be aliquoted, snap frozen in liquid nitrogen and stored at −80° C. until inoculation in the model. Substrates at normal (mean value), high physiologic (2× mean), and supplemental levels (8× mean) will be fed to the microbiota over a period of 3 days after an adaptation period of 16 h through the feeding syringe.

Samples will be taken every 24 h for a period of 72 h from both the lumen and dialysate of the system and analyzed for short chain FAs, fermentation products (e.g., lactate) and microbiota composition (sequencing the V3-V4 region of the 16S rRNA gene using Illumina sequencing).

Objective: We will first examine the difference between human milk and formula on the TIM-2 microbiome. In view of the complexity of human milk, we will subsequently examine the individual effects of potentially modifiable components (lactose, FAs, and individual HMOs).

Methods: Based upon energy requirements of 2 month old males or females, the recommended daily volume of human milk or formula is ˜900 ml.202 Normally ˜10% of lactose remains undigested in the infant gut due to the transit in the upper GI tract. Similarly, approximately 10% of lipids reach the colon. HMOs are not significantly metabolized or absorbed in the upper GI tract. Each study will be tested against a normal control of infant formula and donor human milk, where the assumption is made that approximately 10% of milk volume, lactose and FAs and 100% of HMOs reach the colon. Donor human milk composition will be analyzed, as described above, to assure consistency. Five replicates will be performed for each substrate dose.

Formula: We will administer 90 ml/day donor human milk or Enfamil (Simply Organic, no added HMOs) to the TIM-2, as the control study.

Lactose: Assuming lactose concentration of 7 g/dl and 90% upper GI absorption, we will administer 6.3 g/day lactose, with high physiologic and supplemental doses of 12 and 48 g/day, respectively.

FAs: Assuming hydrolysis in the upper GI tract, but not full absorption, we will administer 3.0 g/day FAs with high physiologic and supplemental doses of 6 and 24 g/day, respectively. Based upon human milk content, individual studies will be performed with medium (lauric and myristic acid) and long chain (palmitic, oleic, and linoleic acid) FAs.

HMOs: Overall, about 70% of HMOs in pooled milk are fucosylated and about 20% arc sialylated. The major components of HMOs arc lacto-N-tetraose (LNT), lacto-N-neotetraose (LNnT), disialylated LNT, as well as monofucosylated, monosialylated, or difucosylated lactose, LNT, and LNnT. We will study the effects of the 10 most abundant HMO species. Each of these HMOs can be synthesized and will be administered in the TIM-2 model at normal, high physiologic and supplemental levels (mean, 2× and 8× mean, respectively).

Expected Results: We chose to study substrates at normal (mean values), high physiologic (2× mean), and supplemental levels (8× mean), as study results may support the use of supplements for breast-fed or formula fed infants. The different treatments (lactose, FA and HMOs) are expected to lead to distinct changes in the gut microbiota (both composition and activity) compared to the control. We expect that HMOs belonging to or derived from type I and type II glycans will lead to an increase in the relative abundance of beneficial taxa, such as bifidobacteria and lactobacilli. We expect these changes in relative abundance to be accompanied by changes in the metabolite profile, with more lactate and acetate produced (the major metabolites of bifidobacteria and lactobacilli). Both lactate and acetate are also precursors for butyrate, the most important fuel for colonocytes, and thus we also expect concentrations of butyrate to be modulated, although this depends on the presence of taxa that can produce butyrate and the extent to which these taxa are affected by the different treatments.

### Example 6. Investigation of Impact of High Fat/High Calorie Milk on Children

This example proposes to investigate the impact of high fat/high calorie milk from women with OW/OB on early life weight gain.

Importance of Breastfeeding: The American Academy of Pediatrics and the WHO recognize exclusive human milk feeding as the “normative standards for infant feeding” for the first six months after birth. Nearly 80% of women initiate breastfeeding at birth and 60% have some breastfeeding continuing at 6 months.

Human Milk Composition: Human milk is a complex biological fluid of more than 200 identified components including solutions, colloids, membranes, globules and live cells. On average, it contains 0.8-0.9% protein, 3-5% fat, 6.9-7.2% carbohydrates, 0.2% minerals and averages 66 kcal/100 mL with an interquartile range 62.0 to 72.5 kcal/100 mL, reflecting the significant individual variance of caloric content. Milk synthesis is a complex process impacted by diet, serum composition, lipogenesis, and substrate uptake and synthesis by mammary epithelial cells (MECs). Although the milk metabolome, proteome, lipidome and small bioactives contribute to infant wellbeing, macronutrients are the primarily determinants of infant caloric intake and growth. Milk carbohydrate is primarily lactose, as well as an array of oligosaccharides. Milk proteins consist of whey (α-lactalbumin, lactoferrin, β-lactoglobulin, albumin and immunoglobulins) and casein (phosphoproteins) in addition to select hormones (leptin, ghrelin). Milk protein and carbohydrate content are remarkably unchanged even in women with OW/OB. As discussed below, milk fat represents the variable component of caloric content.

Fats: Milk fats contribute 40-50% of human milk calories, with triglycerides (TGs) accounting for 98% of milk lipid content. In contrast to lactose and protein, milk fatty acid (FA) composition is highly variable, with both maternal BMI and diet being important determinants of milk polyunsaturated FAs, lipid content and total caloric content. Breast milk fats also increase markedly from foremilk to hindmilk.

Milk fat is primarily contained within milk fat globules (MFG), a triglyceride-rich core surrounded by a tri-layer membrane of protein and lipids, enriched with glycerophospholipids, sphingolipids, cholesterol and protein. Among 400 different milk FAs, only 15 constitute 90% of the total FA pool. In mature human milk, the majority of TGs in the MFG core consist of long chain FAs (LCFAs): [18:1n-9 oleic (20-35%), 16:0 palmitic (18-23%), and 18:2n-6 linoleic (8-18%)]. LC polyunsaturated FAs, notably arachidonic, eicosapentaenoic, and docosahexaenoic acids, are some of the least abundant, though dependent in part on maternal diet and genetics. Medium chain FAs (MCFAs) comprise ˜12% of total FAs, and <1% are short chain FAs (SCFAs) which serve to induce newborn satiety and as critical precursors.

As in humans, mouse milk contains 30-70% LCFAs and 15-40% MCFA (from de novo lipogenesis and preformed FAs), depending on strain and diet. Unlike humans, non-human mammalian milk contains markedly less oligosaccharide. Despite some differences, the mammary gene expression and mammary metabolism, especially the lipogenic system during lactation, is qualitatively very similar in humans and rodents.

Both human and mouse milk TGs result from three sources: endogenous fat stores, dietary lipids, and de novo MEC synthesis. Endogenous fat stores and diet contribute to LCFAs as MECs have limited ability to synthesize C18 FAs. These LCFAs, which are primarily carried by chylomicrons (intestinal absorption) or hepatic very low-density lipoproteins are transported to MECs where apolipoprotein B interacts with MEC surface lipoprotein receptors. Following hydrolysis by MEC lipoprotein lipase (LPL), which increases dramatically with lactation in mice and humans, FAs enter the cell. Depending on the needs of the cells, FAs can be desaturated under the influence of stearoyl-CoA desaturase and/or converted into triglycerides, phospholipids or cholesterol esters.

MEC de novo synthesis contributes primarily SCFAs and MCFAs (C6-C12) with limited production of C14-C16 FAs. Acetate and β-OH butyrate, the primary carbon sources, are absorbed through the MEC basolateral membrane, and synthesis primarily catalyzed by fatty acid synthase (FAS) and acetyl CoA carboxylase (ACACA), while acylthioesterase serves to terminate FA elongation. Among the genes and transcription factors which regulate the complex biosynthesis of milk fat is the family of sterol regulatory element-binding proteins (SREBP) which interact with SREBP cleavage activating protein. The peroxisome proliferator-activated receptor γ also contributes to regulation of fat synthesis in the mammary gland. These targets of MEC FA update and synthesis provide opportunities to regulate milk fat and caloric content.

Milk Transcriptome: The milk fat globule transcriptome reflects the metabolic gene expression profile for the lactating mouse mammary gland, encompassing all aspects of milk FA production, including lipolysis at the MEC membrane, intracellular FA transport, de novo synthesis, elongation and desaturation, as well as TG and cholesterol synthesis and lipid droplet formation. Our studies have confirmed that mRNA is expressed in MEC cell culture secretions in vitro, reflecting MEC cellular activity.

Role of Insulin in Milk Lipid Biosynthesis: As discussed above, lipid synthesis involves the de novo synthesis of FAs as well as the incorporation of de novo and preformed FAs (serum TG origin) into milk triglycerides. Human and mouse studies confirm the significant role of insulin in milk lipid synthesis. The mammary gland becomes highly sensitive to insulin during lactation with its receptor (IR—B) increasing by 2.5-fold. Specific mammary gland IR knockout downregulates an array of genes involved in mice milk lipid synthesis, milk fat globule formation and milk lactose synthesis, with reduced lipid droplets and casein staining. In vitro insulin treatment of mouse MEC cells induces gene expression of transcription factor SREBP1, phosphorylation of Akt and enzymes involved in lipid uptake (LPL) and synthesis (ACACA, FAS).

### Preliminary Data

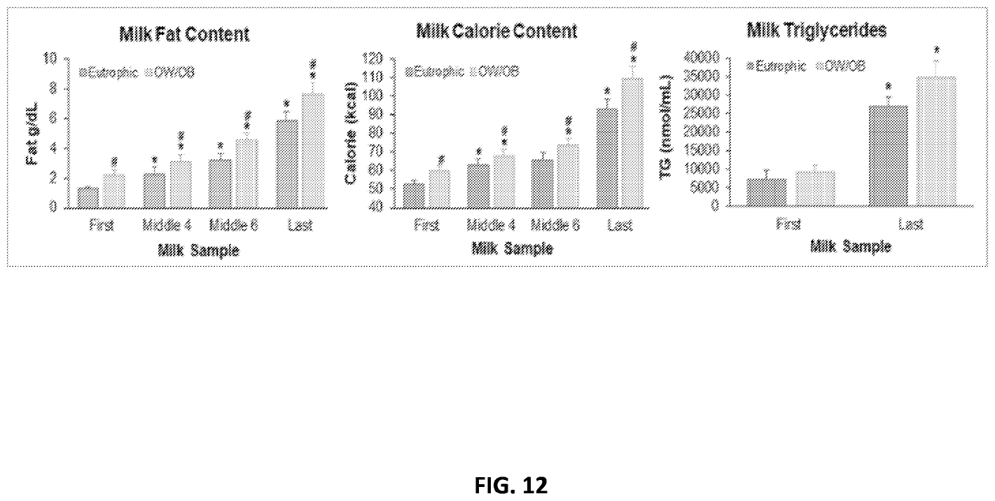

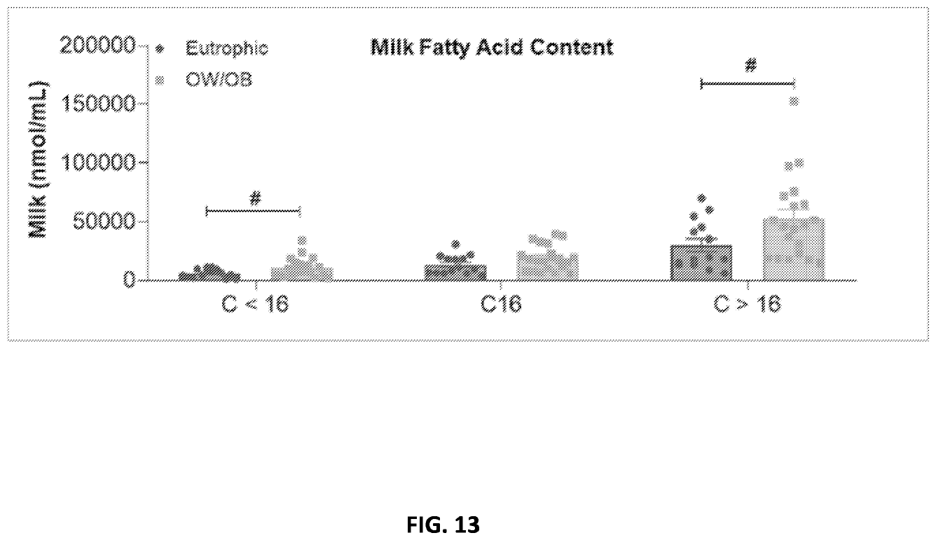

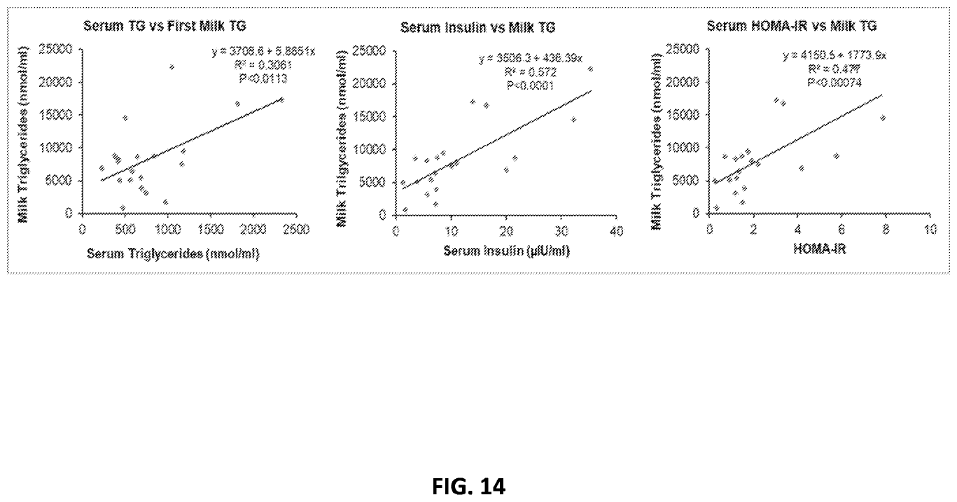

Human Breast Milk Variation in Fat and Caloric Content: We studied women who delivered singleton, term neonates and were exclusive breastfeeding at 7-9 weeks postpartum. Among both eutrophic (BMI 18.5-24.9 kg/m2); and subjects with OW/OB (BMI≥25 kg/m2), continuous breast milk samples (10 ml) from foremilk to hindmilk were obtained via pump until the breast was emptied. Women with OW/OB had a trend towards increased serum insulin levels (13.6±3.4 vs 8.4±2.0, p<0.08). As expected, milk TG, fat and calorie content increased markedly from the first (foremilk) to the last (hindmilk) sample. Women with OW/OB had markedly increased milk fat and calories as well as both MCFAs (<C16) and LCFAs (>C16) (FIG. 12-13). Among all patients, maternal serum TG correlated modestly while maternal serum insulin and HOMA correlated highly with foremilk TG concentration (FIG. 14). These findings emphasize the critical roles of both serum TG and local insulin action on human milk fat content and indicate that the high fat content of OW/O milk is a result of both de novo lipogenesis (MCFAs) and uptake of preformed FAs (LCFAs). Of note, neither serum insulin nor TG levels were predictive of hindmilk TG levels indicating that the regulatory mechanisms of foremilk and hindmilk differ.

Hypothesis: Systemic Insulin Resistance Produces High Fat Milk. Despite its critical importance, there is limited knowledge of the mechanisms contributing to high fat/high calorie content milk in women with OW/OB, and no accepted modalities to modify composition (e.g., maternal diet, supplements, therapeutics) to meet infant needs. Obesity in both humans and mice is associated with increased TGs and insulin levels with systemic insulin resistance. In nondiabetic obese subjects, FA effects of insulin resistance are marked by reduced insulin suppression of adipose free FA release, reduced clearance of adipose tissue and skeletal muscle TG, and increased hepatic lipogenesis, leading to increased postprandial TG concentrations. Importantly, evidence suggests that insulin resistance occurs unequally among insulin target organs (i.e., liver, adipose, muscle). During lactation, there is resistance to insulin stimulation of lipid deposition in adipose tissue, while insulin sensitivity upregulates mammary gland LPL.

Our preliminary studies have prompted a unique hypothesis in which we propose that systemic insulin resistance is a primary cause of high fat milk: Specifically, we postulate that (1) systemic insulin resistance results in increased foremilk TG by causing increased serum TG concentration, while (2) mammary insulin sensitivity stimulates both mammary TG uptake and de novo lipogenesis. Thus, postpartum subjects with insulin resistance have proportionately less insulin resistance (i.e., relative sensitivity) at the lactating breast as compared to the primary insulin target sites. When combined with elevated serum TG concentrations, increased serum insulin stimulates MEC LPL and FAS, promoting insulin-mediated fatty acid uptake and de novo lipogenesis. Together, with insulin-facilitated glucose uptake, which is synthesized to glycerol, insulin will stimulate mammary TG synthesis and incorporation into milk.

Confirmation of this mechanism offers the opportunity to normalize the milk fat concentration in OW/OB insulin-resistant subjects to prevent excessive early life weight gain. We have established mouse MEC cell culture in our laboratory using non-lactating, non-pregnant ICR mice. In culture, MEC cells secrete milk components into the apical compartment from nutrients in the basolateral compartment, while maintaining polarity and tight junctions. We have demonstrated that increased “serum” palmitic increases MEC “milk” β-casein and TG concentrations while MEC cells increased β-casein, FA synthesis proteins (pSTAT5, SREBP, FAS), and LPL with secreted mRNA reflecting an identical pattern of changes in cellular mRNA expression. Most importantly, we demonstrated that inhibition of FAS (with Orlistat) normalized palmitate-induced “milk” TG concentrations to that of untreated MECs, confirming the ability to modulate select enzymatic pathways and milk composition in MEC culture.

This example aims to address the major pathway of the origin of milk fat and potential ways to alleviate this. These studies use a combined approach of a novel 3-dimensional MEC culture and an in vivo obese mouse model to understand the putative mechanisms regulating mammary TG uptake and FA synthesis, and the pathway(s) by which insulin resistance results in high fat/high calorie milk composition. In vitro studies will modify “serum” TG concentration and interrogate select insulin-specific cellular pathways to develop strategies to modulate milk FAs. In vivo studies will test the effect of metformin-increased insulin sensitivity to assess the impact on milk fat composition and to confirm the normalization of newborn growth during nursing. These preclinical studies are essential for the ultimate development of mechanism-based therapies to optimize human milk composition for personalized infant nutrition, specifically focused on normalizing the fat and caloric of milk for infants at risk of excessive weight gain.

### To Confirm the Impact and Mechanisms of Increased “Serum” Substrates and Insulin on MEC Milk Fat Content

FA substrates and insulin independently and synergistically regulate milk fat composition. Specifically, we propose that (1) Increased serum acetate and TGs will upregulate mammary de novo lipogenesis and FA uptake, respectively, (2) Increased insulin will stimulate both de novo lipogenesis and FA uptake, and (3) Increased serum TGs and insulin will synergistically increase milk fat content.

Methods: Mouse MEC 3D culture: Non-pregnant ICR mice will be utilized for MEC culture as described.

Sample Collection: Mouse mammary tissue samples will be obtained and prepared in well inserts with lactation medium (LM) in basolateral chamber and RPMI in the apical (“milk”) chamber. After 2 days, test substrates or pharmacologic agents are added to the basolateral chamber. After 48 h of treatment, MECs (cells) and apical culture medium (“milk”) will be collected for analysis.

Reagents: Glucose free RPMI 1640 medium (#11879020, Thermo Fisher), lipid and amino acid depleted/dialyzed FBS (Neuromics, Edina, MN), Amino Acid Mixture (Promega, USA) and customized (mouse) TG mixture (Nu-Chek Prep Inc., Elysian, MN) containing 30% saturated fat (15% palmitic acid, 15% stearic acid, 7% palmitoleic acid, 26% oleic acid, 20% linoleic acid, 16% linolenic acid, 1% EPA.

Growth Medium (GM): Glucose free RPMI 1640 with added 5 mmol/L glucose, 10% (% v/v) lipid depleted FBS supplemented with Normal TG (as above), 0.5 mM amino acid mixture, 0.1 ng/mL insulin, 10 ng/mL EGF, 14.0 μg/mL phosphatidylethanolamine.

Lactogenic Medium (LM): Glucose free RPMI 1640 with added 5 mmol/L glucose, 10% (% v/v) lipid depleted FBS, 0.5 mM amino acid mixture, 10 ng/ml EGF, 14.0 μg/mL phosphatidylethanolamine, 0.5 U/mL of prolactin and 1 μM dexamethasone. Acetate, TG and insulin will be added as specified below. All TG mixtures will be at 30% saturated fat.

### Availability of “Serum” FA Substrates and Insulin Alters Milk Fat Composition

Hypothesis: We propose that substrate-dependent “serum” FA components alter milk fat content via either de novo lipogenesis and/or TG uptake. We propose that insulin potentiates the effect of increased serum FAs on milk fat content via both pathways.

Methods: In all studies, the basolateral chamber will contain LM, and MECs will be cultured for 48 h.

“Serum” acetate and TG concentrations in “controls” will be set at mean serum concentrations with low and high physiologic concentrations sct at 50% and 200% of the mean, respectively, and insulin at levels from normal to that observed in obese mice.

### Substrate Modulation:

SCFA and MCFA: LM will be supplemented with sodium acetate (0.05, 0.1, 0.2 mmol/L) in the presence and absence of normal TG concentration (100 mg/dl).

TGs and LCFA: LM will be supplemented with lipid depleted FBS supplemented with TG (50, 100 and 200 mg/dL) in the presence and absence of normal acetate concentration (0.1 mmol/L).

Hormone Modulation: Insulin: LM will be supplemented with insulin (0.5, 2 and 10 ng/ml) in the presence and absence of normal TG and acetate concentrations.

Substrate and Insulin Modulation: Both insulin and TG/acetate concentrations will be modified to examine synergistic effects.

Interrogation of Insulin-Stimulated Pathways which Increase MEC Lipid Synthesis and Milk Fat Production

Hypothesis: We propose that (1) insulin-stimulated pathways will increase milk fat by increasing TG uptake and inducing key de novo lipogenic genes, and (2) pharmacologic modulation of insulin pathways will predictably alter “milk” composition.

Insulin stimulation of insulin receptors (IR) activates PI3K/Akt signaling pathway that activates transcription factor SREBP1 and its downstream targets resulting in increased mammary LPL, FAS and ACACA. We will interrogate the pathway-specific effects of insulin on the stimulation of MEC milk fat production. We will silence (1) TG uptake enzyme Lpl, (2) de novo lipogenic enzymes Fas and Acaca and (3) IR which will inhibit both pathways. We will also utilize specific pharmacologic agents targeting LPL and FAS to confirm the critical role of insulin in mammary lipid synthesis.

Methods: MEC cells will be cultured as described above. In all studies, MEC cells will be treated with insulin (normal and high; 2.0, 10 ng/ml) and studied in LM supplemented with TG (100 mg/dl) and acetate (0.1 mmol/L) dependent upon Study 1A results).

Transfected and pharmacologic treated MECs will be analyzed as detailed below.

MEC Cell Analysis: MEC cell viability (MTT assay), intact barrier function (bidirectional flux of FITC-dextran 3000) and transepithelial resistance (Millicell-ERS system, Millipore) will be confirmed. Milk synthesis will be confirmed by immunostaining for MEC cellular β-casein (milk), BODIPY (lipids) and adipophilin. MEC “Milk” Composition: Upper medium (“milk”) will be collected for analysis for lactose (enzymatic assay Cat. #MAK017, Sigma-Aldrich), glucose (glucose oxidase activity assay #MAK097, Sigma), β-casein (ELISA assay #MBS7229565, MyBioSource, Inc, San Diego, CA) and lipids (Sciex 5500 with DMS device, Lipidyzer Platform, UCLA Lipidomics). MEC cells and “milk” gene expression: MEC cellular and secreted mRNA will specify the impact of modified substrates, target pathway modulators and pharmacologic interventions on MEC FA pathways. These mRNA responses will be applied to in vivo (and future clinical studies) to confirm impact of interventions on mouse milk composition and newborn growth. RNA-Seq (RNA isolation, quality evaluation, RNA-Seq and bioinformatics) will be performed at Technology Center for Genomics & Bioinformatics (TCGB) Core, UCLA. We will focus the analysis on the 456 genes found to be associated with lactation. Those genes that show significant change will further be confirmed by RT-PCR.

Expected Results/Problems/Future Studies: An initial dose response curve will be performed to confirm physiologic and pharmacologic insulin concentrations which impact milk fat production. We expect increased acetate and insulin, including Lpl silencing will upregulate de novo lipogenesis enzymes and increase MCFAs. At high TG concentrations and silencing of Fas and Acaca, we expect increased LCFAs correlated with increased FA uptake, intracellular FA transporters, and decreased expression of de novo SCFA synthesis. We further expect that silencing of IR will downregulate uptake as well as de novo lipogenesis. Whilst treatment with NDGA and Cerulenin will reduce “Milk” fat, the former would specifically decrease LCFAs whereas the latter will reduce MCFAs. We expect that higher insulin will potentiate the effects of increased TG concentration. In all cases, we expect that MEC “milk” gene expression will correlate with that of MEC cells.

### Enhancement of Systemic Insulin Sensitivity Will Reduce Obese Mouse Milk Fat Content and Normalize Newborn Growth

Metformin improves insulin sensitivity by increasing insulin-mediated insulin receptor tyrosine kinase activity. In addition to antihyperglycemic properties, metformin lowers plasma TGs. We propose that metformin administration to obese mice will reduce milk fat content via 1) a reduction in plasma TGs, and 2) a reduction in serum insulin levels with proportionately less mammary tissue insulin effect. We propose that decreased uptake and decreased MEC de novo lipogenesis will lower the milk fat content.

Methods: All studies will utilize weanling female ICR fed either a high fat (45% Kcal; Research Diet D12451, New Brunswick, NJ) to create obese dams or standard diet (10% Kcal fat; Research Diet D12450B, New Brunswick, NJ), mated at 11 weeks of age, and maintained on the same diet during pregnancy and lactation. All mice will be housed at 22° C. with regulated humidity and a controlled 12:12 h light/dark cycle. Following birth, litter size is standardized to 3 males and 3 females and all offspring weaned to standard fat (10% Kcal) diet.

Increased Systemic Insulin Sensitivity will Reduce Obese Mouse Milk Fat Content

Hypothesis: Metformin-induced increased systemic insulin sensitivity will both reduce serum triglycerides and serum insulin, and reduce milk FA content. Milk secreted mRNA will reflect modulation of critical MEC pathways.

Methods: On day 2 of lactation, obese (OB) and control (Con) dams will be randomly divided into two groups with free access to drinking water with or without metformin (S1950, Selleck, TX) (250 mg/kg/day, equivalent to human dose of 20 mg/kg/d), resulting in four groups: OB, OB-Met, Con and Con-Met.

Dose response studies will be performed to achieve a 10% reduction in milk FA concentration. Prior to treatment, blood via tail vein and milk sample will be collected. Following treatment, milk samples will be collected at postnatal day 12, 16 and 20. We have previously shown rapid growth rate of pups from obese/high fat diet fed dams during this period. At the end of nursing period (p20), maternal blood will be collected (cardiac puncture).

Milk Collection: Dams and their pups will be separated for 5 hours, after which physical contact will be re-established for 5 mins in order to naturally stimulate milk ejection. Dams will then be lightly anesthetized (isoflurane; oxygen) followed by administration of 0.1 ml oxytocin (2 IU, ip). The nipple area will be moistened with sterilized water and milk collected using Tygon tubing with 200-p pipette tip connected to an electric pump (Swing Breast Pump, Medela). From each dam, milk samples will be collected from all teats and pooled. Typically, 350-400 μl milk per mouse can be collected using this method. Milking will be collected at a fixed time (between 11.00 and 12 hours) to avoid diurnal rhythm changes.

Analysis: Dams: Daily water intake and body weight of dams will be recorded and metformin dose will be calculated. Additionally, serum metformin levels will be analyzed by LC-MS/MS system at UCLA Mass Spectrometry Laboratory Core. Serum samples will be analyzed for lipids, glucose and insulin levels. Mammary glands will be stained for lipid and β-casein. Milk: Lactose, glucose, lipids, β-casein and calorie (Bomb calorimetry) content, including mRNA gene expression will be determined as stated above. For RNA Seq, milk fat layer will be isolated by centrifugation (1,000 g for 5 min) at 4° C. and analyzed at TCGB UCLA core.

### Reduction of Milk Fat Content Will Normalize Growth of Offspring of Obese Dams

Hypothesis: We propose that reduction in breast milk fat content will prevent the excessive newborn weight gain in offspring of obese dams.

Methods: Beginning on day 2 of lactation, obese and control dams with be treated with metformin, (as described above) or control water. Weekly body weight will be recorded. On day 20 male and female mice will be weighed and fat/lean body mass assessed by DEXA and blood collected for TG, lipids, glucose and insulin levels. To assess long-term effects, offspring will be weaned to normal diet and followed. Offspring at 12 weeks of age will undergo DEXA and GTT after which blood will be collected via cardiac puncture and euthanized.

Expected Results/Problems/Future Studies: We expect that metformin will reduce milk fat and caloric content, with pathway specific modulation reflected by secreted mRNA.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for improving the quality of breast milk in a female human individual that provides breast milk, or expects to provide breast milk, to a child, comprising administering to the individual an agent that increases the individual's sensitivity to insulin.

2. The method of claim 1, wherein the agent is selected from the group consisting of:
metformin;
rosiglitazone;
pioglitazone;
D-chiro-inositol;
catechin;
N-acetyl cysteine;
an anti-CRFR2 antibody; and
an anti-Fyn antibody.

3. The method of claim 1, wherein the agent is metformin.

4. The method of claim 1, wherein the individual does not have diabetes.

5. A method for improving the quality of breast milk in a female human individual that provides breast milk, or expects to provide breast milk, to a child, comprising administering to the individual an agent that:
(a) reduces the substrate uptake, synthesis or secretion of long chain fatty acids;
(b) reduces the substrate uptake, synthesis or secretion of short chain fatty acids;
(c) increases the amino acid uptake, protein synthesis or protein secretion of proteins; or
(d) reduces the uptake or synthesis of lactose.

6. The method of claim 5, wherein the agent:
(a) inhibits the expression or activity of lipoprotein lipase, fatty acid synthase, SREBP, PPAR or LCFA;
(b) inhibits the expression or activity of acetyl COA, hydroxybutyrate, acetyl COA carboxylase, acylthioesterase, or MCFA;
(c) increases the expression or activity of amino acid transporter, mTOR, casein, or lactalbumin; or
(d) inhibits the expression or activity of GLUT1, UDP-galactose, lactose synthase, or α-lactalbumin.

7. The method of claim 5, wherein the agent is selected from the group consisting of orlistat, omeprazole, linoleic acid or conjugated linoleic acid, GW9662, CP-640186, a growth hormone, leucine, methionine, phlorizin, phloretin, theophylline, progesterone, and inhibitory RNA molecules.

8. The method of claim 1, wherein the individual is overweight.

9. The method of claim 1, wherein the individual has a BMI >20, >25, or >30, >35, >40 or >50.

10. The method of claim 1, wherein the individual is pregnant.

11. The method of claim 1, wherein the individual has given birth within the previous 2 years.

12. The method of claim 11, wherein the individual is breastfeeding.

13. The method of claim 1, wherein the agent is administered orally.

14. The method of claim 1, wherein the agent is injected to the breast.

15. The method of claim 1, wherein the child is overweight.

16. The method of claim 15, wherein the child has a BMI >20, >25, or >30, >35, >40 or >50.

17. The method of claim 1, wherein the agent is administered daily, once every 2 days, 3 days, 4 days, 5 days, 6 days, 7 days, 2 weeks, 3 weeks or 4 weeks.

18. The method of claim 1, wherein the individual is not otherwise prescribed for administration of the agent.

19. The method of claim 1, wherein the agent is administered at a dose or frequency, or both, for when being used for treating another indication.

20. The method of claim 1, further comprising administering to the individual a second agent that:
(a) reduces the substrate uptake, synthesis or secretion of long chain fatty acids;
(b) reduces the substrate uptake, synthesis or secretion of short chain fatty acids;
(c) increases the amino acid uptake, protein synthesis or protein secretion of proteins; or
(d) reduces the uptake or synthesis of lactose.
